(19) United States
(12) Patent Application Publication
(10) Pub. No.: US 2013/0004840 A1
(43) Pub. Date: Jan. 3, 2013

(54) NON-AQUEOUS ELECTROLYTE ADDITIVE FOR LITHIUM SECONDARY BATTERY, NON-AQUEOUS ELECTROLYTE, AND LITHIUM SECONDARY BATTERY INCLUDING THE SAME

(76) Inventors: Jung-Yi YU, Yongin-si (KR); Woo-Cheol Shin, Yongin-si (KR); Sang-Il Han, Yongin-si (KR); Mi-Hyun Lee, Yongin-si (KR); Tae-Hyun Bae, Yongin-si (KR)

(21) Appl. No.: 13/363,528

(22) Filed: Feb. 1, 2012

(30) Foreign Application Priority Data
Jun. 30, 2011 (KR) 10-2011-0065111

(51) Int. Cl. H01M 10/056; C07F 9/02; C07C 53/08

(52) U.S. Cl. 429/203; 560/55; 568/15; 429/188

(57) ABSTRACT

A non-aqueous electrolyte additive for a lithium secondary battery, a non-aqueous electrolyte for a lithium secondary battery, and a lithium secondary battery, the additive including a bidentate phosphorus compound represented by the following Chemical Formula 1.

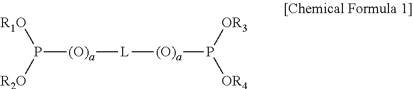

### BACKGROUND

1. Field

Embodiments relate to a non-aqueous electrolyte additive for a lithium secondary battery, a non-aqueous electrolyte, and a lithium secondary battery including the same.

2. Description of the Related Art

As portable electronic and communication devices, e.g., a video camera, a cellular phone, a laptop, and the like, become smaller and lighter, a battery as a power source for these devices should have high energy density, and should be small and light. A lithium secondary battery may include an organic electrolyte, and may have two times or more discharge voltage than a lithium secondary battery including an alkali aqueous solution, and thus may have a high energy density. Accordingly, a lithium secondary battery may be smaller and lighter, and may have high-capacity charge and discharge.

### SUMMARY

Embodiments are directed to a non-aqueous electrolyte additive for a lithium secondary battery, a non-aqueous electrolyte, and a lithium secondary battery including the same.

The embodiments may be realized by providing a non-aqueous electrolyte additive for a lithium secondary battery, the additive including a bidentate phosphorus compound represented by the following Chemical Formula 1,

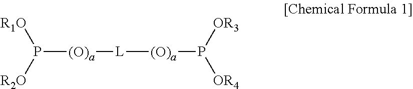

wherein a is independently 0 or 1, and R₁, R₂, R₃, and R₄ are each independently selected from the group of a substituted or unsubstituted C1 to C15 alkyl group, a substituted or unsubstituted C3 to C15 alkylsilyl group, a substituted or unsubstituted C3 to C15 cycloalkyl group, a substituted or unsubstituted C3 to C15 heterocycloalkyl group, a substituted or unsubstituted C6 to C15 aryl group, a substituted or unsubstituted C2 to C15 heteroaryl group, and a substituted or unsubstituted C2 to C15 alkenyl group, or R₁, R₂, R₃ and R₄ are independently linked together to provide one selected from the group of an oxygen-containing substituted or unsubstituted C2 to C15 heterocycloalkyl group and an oxygen-containing substituted or unsubstituted C2 to C15 heteroaryl group, and L is selected from the group of a C2 to C15 alkenylene group, a covalent bond bridging group represented by the following Chemical Formula 2, and a covalent bond bridging group represented by the following Chemical Formula 3,

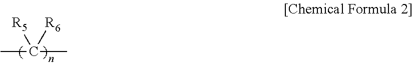

wherein n is an integer of 1 to about 4, and R₅ and R₆ are linked together to provide one selected from the group of a substituted or unsubstituted C3 to C15 cycloalkyl group, a substituted or unsubstituted C3 to C15 heterocycloalkyl group, a substituted or unsubstituted C3 to C20 aryl group, and a substituted or unsubstituted C2 to C20 heteroaryl group, or R₅ and R₆ are each independently selected from the group of hydrogen, an amine group, a cyano group, a thiolate group, a substituted or unsubstituted C1 to C15 alkyl group, —OR₇, —SiR₈R₉R₁₀, —OSiR₁₁R₁₂R₁₃, —SR₁₄, —SOR₁₅, —BR₁₆R₁₇, —OBR₁₈R₁₉, —OCOR₂₀ and —COR₂₁, in which R₇ to R₁₉ are each independently a substituted or unsubstituted C1 to C15 alkyl group, a substituted or unsubstituted C3 to C15 cycloalkyl group, a substituted or unsubstituted C3 to C15 heterocycloalkyl group, a substituted or unsubstituted C6 to C15 aryl group, or a substituted or unsubstituted C2 to C15 heteroaryl group,

(CH₂)_{b}-(A,X)Ar(Y,D)-(CH₂)_{b} [Chemical Formula 3]

wherein b is independently 0 to about 6, Ar is a substituted or unsubstituted C6 to C30 aryl group or a substituted or unsubstituted C1 to C30 heteroaryl group, and A, X, Y, and D are a substituent of Ar, and are each independently selected from the group of hydrogen, a substituted or unsubstituted C1 to C15 alkyl group, a substituted or unsubstituted C3 to C15 cycloalkyl group, a substituted or unsubstituted C3 to C15 heterocycloalkyl group, a substituted or unsubstituted C6 to C20 aryl group, a substituted or unsubstituted C2 to C20 heteroaryl group, a substituted or unsubstituted C2 to C15 alkenyl group, a substituted or unsubstituted C2 to C15 alkynyl group, a halo group, a cyano group, a nitro group, —OH, —OR₂₀, —COR₂₁, —COOR₂₂, —SH, —SR₂₃, —COSR₂₄, —NH₂, —NHR₂₅, —NR₂₆R₂₇, —NO₂, —CN, —CONH₂, —CONHR₂₈, and —CONR₂₉R₃₀, in which R₂₀ to R₃₀ are each independently a C1 to C15 alkyl group.

The compound represented by Chemical Formula 1 may form a cyclic chelate with a metal ion eluted from a positive electrode for a lithium secondary battery including the additive.

The compound represented by Chemical Formula 1 may include at least one of 1,3-bis(diphenoxyphosphine)propane, 1,3-bis(di(trimethylsiloxy)phosphine)methane, bis[1,3,2-dioxaphospholanyl-2]methane, 1,3-bis-[1,3,2-dioxaphospholanyl-2]propane, 1,2-bis[di(t-butoxy)phosphinomethyl]benzene, 1,2-bis[di(trimethylsiloxy)phosphinomethyl]benzene, 1,2-bis[di(t-butoxy)phosphinomethyl]-3,6-diphenyl-4,5-dimethylbenzene, 1,2-bis[di(t-butoxy)phosphinomethyl]-4,5-diphenylbenzene, 1,2-bis(decyloxy phosphorous acid)ethane, 1,4-bis(di(trimethylsiloxy)phosphino)-1,4-dioxabutane, phosphorous acid 2-(diphenoxy-phosphanyloxy)-ethyl ester diphenyl ester, phosphorous acid diallyl ester 2-(bis-allyloxy-phosphanyloxy)-ethyl ester, 1,4-bis(1,3,2-dioxaphosphacyclopentyl)-1,4-dioxabutane), 2-[2-(1,3-dioxa-6-aza-2-phospha-phenalen-2-yloxy)-ethoxy]-1,3-dioxa-6-aza-2-phospha-phenalene), phosphorous acid 2-amino-2-(diethoxy-phosphanyloxy)-ethyl ester diethyl ester, phosphorous acid 2-cyano-2-(diethoxy-phosphanyloxy)-ethyl ester diethyl ester, phosphorous acid 2-(diethoxy-phosphanyloxy)-2-mercapto-ethyl ester diethyl ester, phosphorous acid 2-(diethoxy-phosphanyloxy)-butyl ester diethyl ester, phosphorous acid 2-(diethoxy-phosphanyloxy)-1-methyl-propyl ester diethyl ester, phosphorous acid 2-(diethoxy-phosphanyloxy)-2-ethoxy-ethyl ester diethyl ester, phosphorous acid 2-(tert-butyl-dimethyl-silanyl)-2-(diethoxy-phosphanyloxy)-ethyl ester diethyl ester, phosphorous acid 2-(diethoxy-phosphanyloxy)-2-trimethylsilanyloxy-ethyl ester diethyl ester, phosphorous acid 2-(diethoxy-phosphanyloxy)-2-ethylsulfanyl-ethyl ester diethyl ester, phosphorous acid 2-(diethoxy-phosphanyloxy)-2-(4,4,5,5-tetramethyl-[1,3,2]dioxaborolan-2-yl)-ethyl ester diethyl ester, carbonic acid 1,2-bis-(diethoxy-phosphanyloxy)-ethyl ester methyl ester, acetic acid 1,2-bis-(diethoxy-phosphanyloxy)-ethyl ester, and compounds represented by the following Chemical Formulas 11 to 46, in which iPr denotes isopropyl,

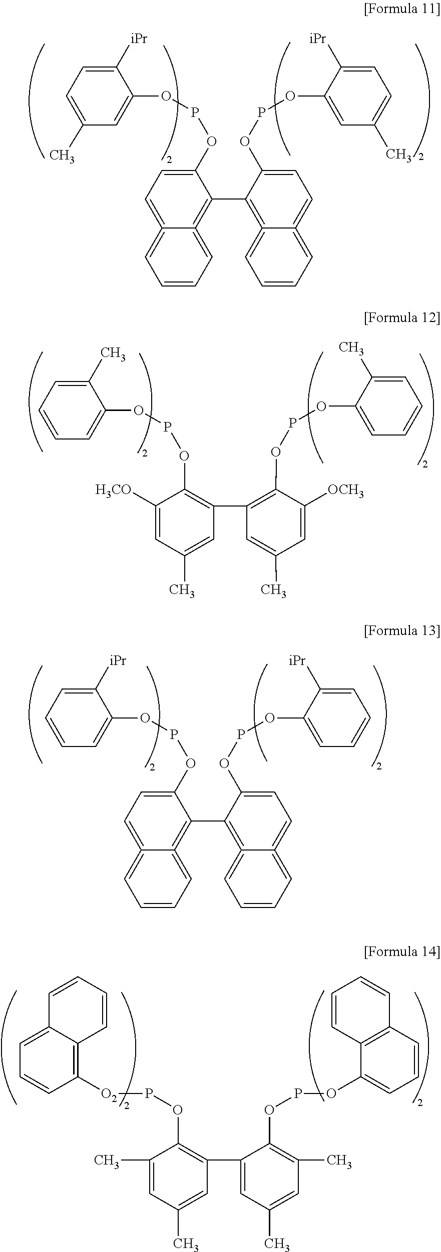

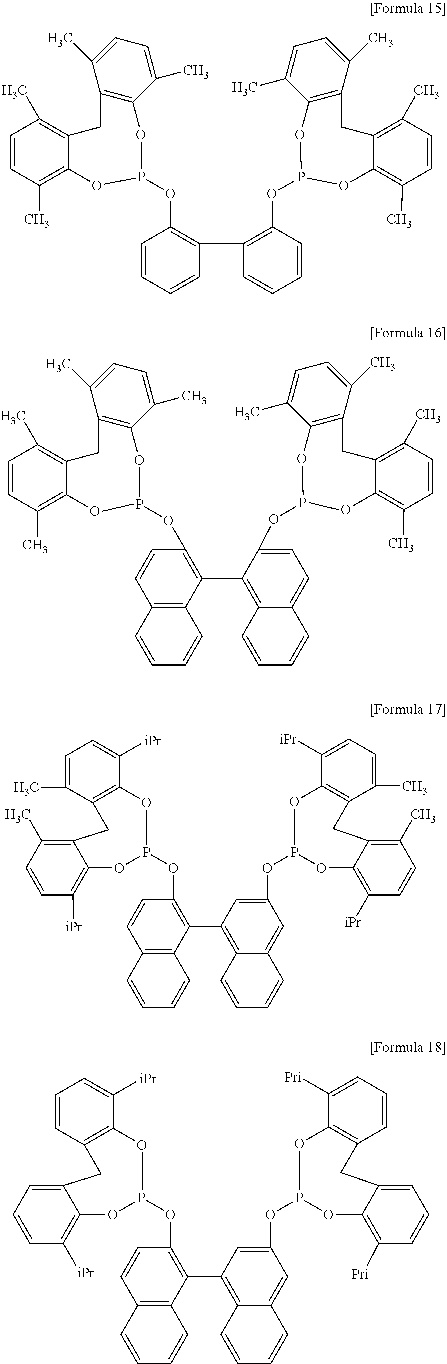

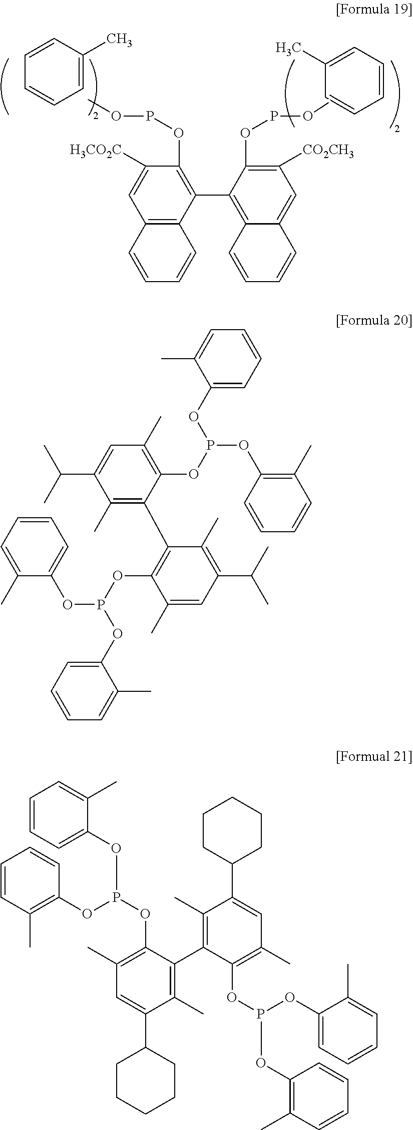

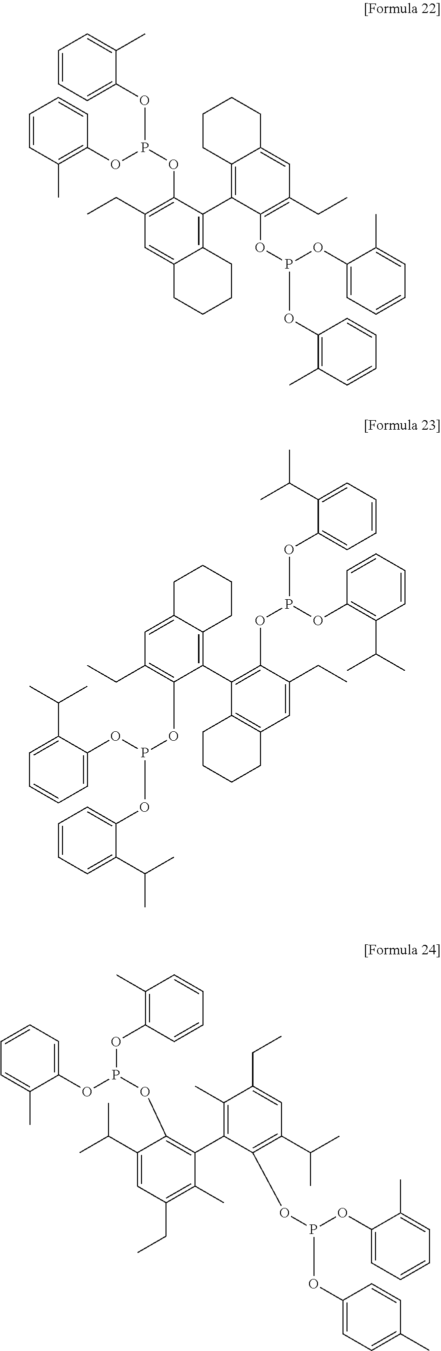 [SMILES: CCc1cc2c(c(-c3c4c(cc(CC)c3OP(Oc3ccccc3C)Oc3ccccc3C)CCCC4)c1OP(Oc1ccccc1C)Oc1ccccc1C)CCCC2 ; CCc1cc2c(c(-c3c4c(cc(CC)c3OP(Oc3ccccc3C(C)C)Oc3ccccc3C(C)C)CCCC4)c1OP(Oc1ccccc1C(C)C)Oc1ccccc1C(C)C)CCCC2 ; CCc1cc(C(C)C)c(OP(Oc2ccc(C)cc2)Oc2ccccc2C)c(-c2c(C)c(CC)cc(C(C)C)c2OP(Oc2ccccc2C)Oc2ccccc2C)c1C]

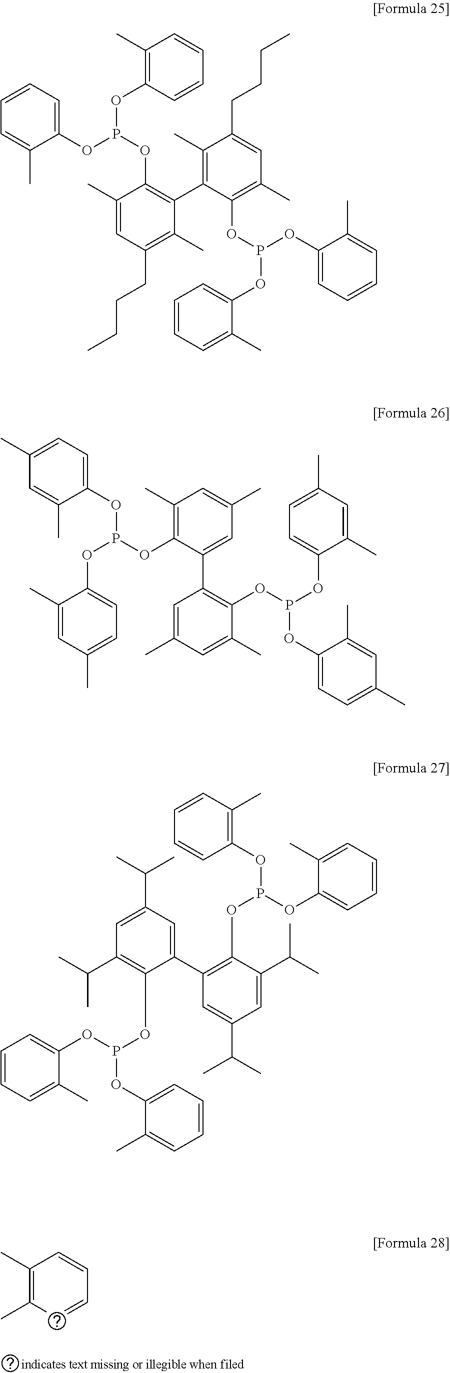

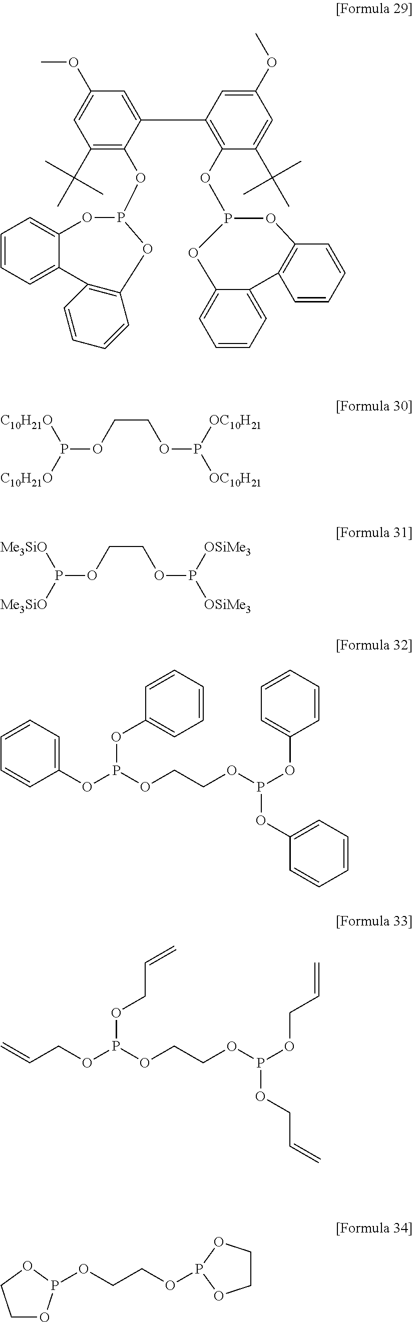

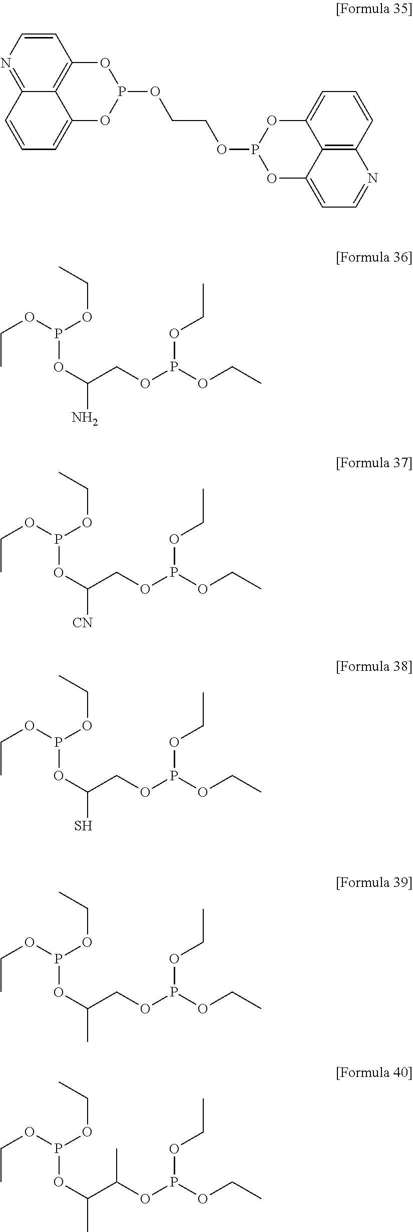

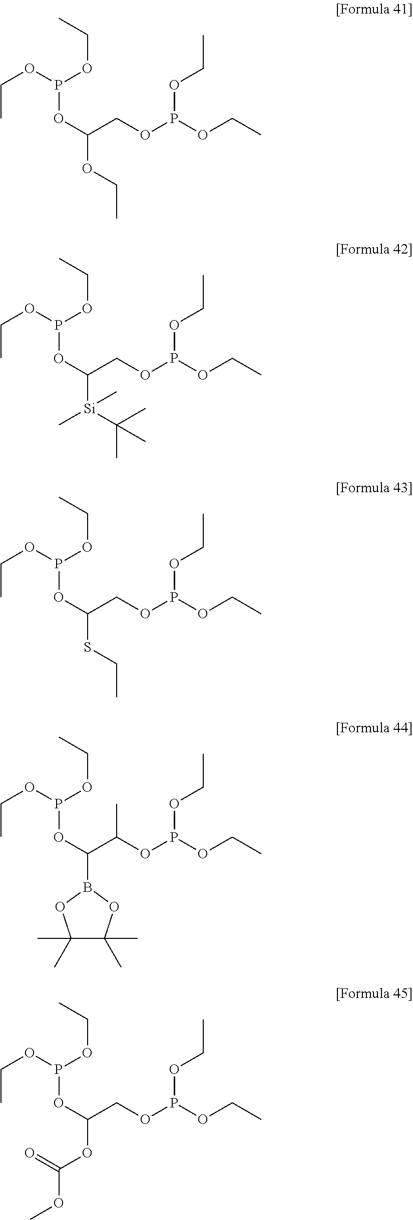

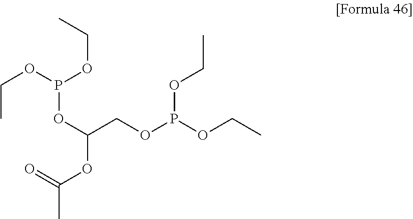 [SMILES: CCOP(OCC)OCC(OC(C)=O)OP(OCC)OCC]

The compound represented by Chemical Formula 1 may include at least one of 6,6′-[(3,3′-di-t-butyl)-5,5′-dimethoxy-1,1′-biphenyl-2,2′-diyl)bis(oxy)]bis(dibenzo[d,f][1,3,2]dioxaphosphepin)hemi ethyl acetate) and 1,4-bis(1,3,2-dioxaphosphacyclopentyl)-1,4-dioxabutane.

The embodiments may also be realized by providing a non-aqueous electrolyte for a lithium secondary battery, the electrolyte including a base electrolyte including a non-aqueous organic solvent and a lithium salt dissolved in the non-aqueous organic solvent; and a bidentate phosphorus compound represented by the following Chemical Formula 1.

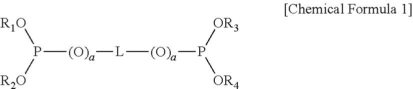

wherein a is independently 0 or 1, and R₁, R₂, R₃, and R₄ are each independently selected from the group of a substituted or unsubstituted C1 to C15 alkyl group, a substituted or unsubstituted C3 to C15 alkylsilyl group, a substituted or unsubstituted C3 to C15 cycloalkyl group, a substituted or unsubstituted C3 to C15 heterocycloalkyl group, a substituted or unsubstituted C6 to C15 aryl group, a substituted or unsubstituted C2 to C15 heteroaryl group, and a substituted or unsubstituted C2 to C15 alkenyl group, or R₁, R₂, R₃ and R₄ are independently linked together to provide one selected from the group of an oxygen-containing substituted or unsubstituted C2 to C15 heterocycloalkyl group and an oxygen-containing substituted or unsubstituted C2 to C15 heteroaryl group, and L is selected from the group of a C2 to C15 alkenylene group, a covalent bond bridging group represented by the following Chemical Formula 2, and a covalent bond bridging group represented by the following Chemical Formula 3,

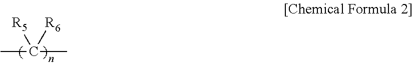

wherein n is an integer of 1 to about 4, and R₅ and R₆ are linked together to provide one selected from the group of a substituted or unsubstituted C3 to C15 cycloalkyl group, a substituted or unsubstituted C3 to C15 heterocycloalkyl group, a substituted or unsubstituted C3 to C20 aryl group, and a substituted or unsubstituted C2 to C20 heteroaryl group, or R₅ and R₆ are each independently selected from the group of hydrogen, an amine group, a cyano group, a thiolate group, a substituted or unsubstituted C1 to C15 alkyl group, —OR₇, —SiR₈R₉R₁₀, —OSiR₁₁R₁₂R₁₃, —SR₁₄, —SOR₁₅, —BR₁₆R₁₇, —OBR₁₈R₁₉, —OCOR₂₀ and —COR₂₁, in which R₇ to R₁₉ are each independently a substituted or unsubstituted C1 to C15 alkyl group, a substituted or unsubstituted C3 to C15 cycloalkyl group, a substituted or unsubstituted C3 to C15 heterocycloalkyl group, a substituted or unsubstituted C6 to C15 aryl group, or a substituted or unsubstituted C2 to C15 heteroaryl group,

(CH₂)_{b}-(A,X)Ar(Y,D)-(CH₂)_{b} [Chemical Formula 3]

wherein b is independently 0 to about 6, Ar is a substituted or unsubstituted C6 to C30 aryl group or a substituted or unsubstituted C1 to C30 heteroaryl group, and A, X, Y, and D are a substituent of Ar, and are each independently selected from the group of hydrogen, a substituted or unsubstituted C1 to C15 alkyl group, a substituted or unsubstituted C3 to C15 cycloalkyl group, a substituted or unsubstituted C3 to C15 heterocycloalkyl group, a substituted or unsubstituted C6 to C20 aryl group, a substituted or unsubstituted C2 to C20 heteroaryl group, a substituted or unsubstituted C2 to C15 alkenyl group, a substituted or unsubstituted C2 to C15 alkynyl group, a halo group, a cyano group, a nitro group, —OH, —OR₂₀, —COR₂₁, —COOR₂₂, —SH, —SR₂₃, —COSR₂₄, —NH₂, —NHR₂₅, —NR₂₆R₂₇, —NO₂, —CN, —CONH₂, —CONHR₂₈, and —CONR₂₉R₃₀, in which R₂₀ to R₃₀ are each independently a C1 to C15 alkyl group.

The compound represented by Chemical Formula 1 may form a cyclic chelate with a metal ion eluted from a positive electrode of lithium secondary battery including the non-aqueous electrolyte additive.

The compound represented by Chemical Formula 1 may be included in the electrolyte in an amount of about 0.01 wt % to about 20 wt %, based on a total weight of the base electrolyte.

The compound represented by Chemical Formula 1 may include at least one of 1,3-bis(diphenoxyphosphine)propane, 1,3-bis(di(trimethylsiloxy)phosphine)methane, bis[1,3,2-dioxaphospholanyl-2]methane, 1,3-bis-[1,3,2-dioxaphospholanyl-2]propane, 1,2-bis[di(t-butoxy)phosphinomethyl]benzene, 1,2-bis[di(trimethylsiloxy)phosphinomethyl]benzene, 1,2-bis[di(t-butoxy)phosphinomethyl]-3,6-diphenyl-4,5-dimethylbenzene, 1,2-bis[di(t-butoxy)phosphinomethyl]-4,5-diphenylbenzene, 1,2-bis(decyloxy phosphorous acid)ethane, 1,4-bis(di(trimethylsiloxy)phosphino)-1,4-dioxabutane, phosphorous acid 2-(diphenoxy-phosphanyloxy)-ethyl ester diphenyl ester, phosphorous acid diallyl ester 2-(bis-allyloxy-phosphanyloxy)-ethyl ester, 1,4-bis(1,3,2-dioxaphosphacyclopentyl)-1,4-dioxabutane), 2-[2-(1,3-dioxa-6-aza-2-phospha-phenalen-2-yloxy)-ethoxy]-1,3-dioxa-6-aza-2-phospha-phenalene), phosphorous acid 2-amino-2-(diethoxy-phosphanyloxy)-ethyl ester diethyl ester, phosphorous acid 2-cyano-2-(diethoxy-phosphanyloxy)-ethyl ester diethyl ester, phosphorous acid 2-(diethoxy-phosphanyloxy)-2-mercapto-ethyl ester diethyl ester, phosphorous acid 2-(diethoxy-phosphanyloxy)-butyl ester diethyl ester, phosphorous acid 2-(diethoxy-phosphanyloxy)-1-methyl-propyl ester diethyl ester, phosphorous acid 2-(diethoxy-phosphanyloxy)-2-ethoxy-ethyl ester diethyl ester, phosphorous acid 2-(tert-butyl-dimethyl-silanyl)-2-(diethoxy-phosphanyloxy)-ethyl ester diethyl ester, phosphorous acid 2-(diethoxy-phosphanyloxy)-2-trimethylsilanyloxy-ethyl ester diethyl ester, phosphorous acid 2-(diethoxy-phosphanyloxy)-2-ethylsulfanyl-ethyl ester diethyl ester, phosphorous acid 2-(diethoxy-phosphanyloxy)-2-(4,4,5,5-tetramethyl-[1,3,2]dioxaborolan-2-yl)-ethyl ester diethyl ester, carbonic acid 1,2-bis-(diethoxy-phosphanyloxy)-ethyl ester methyl ester, acetic acid 1,2-bis-(diethoxy-phosphanyloxy)-ethyl ester, and compounds represented by the following Chemical Formulas 11 to 46, in which iPr denotes isopropyl,

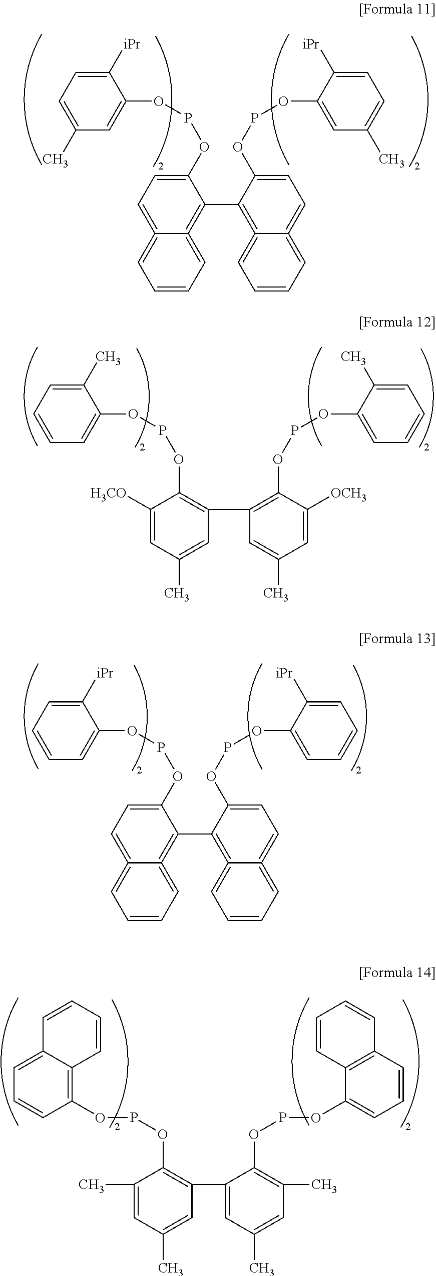

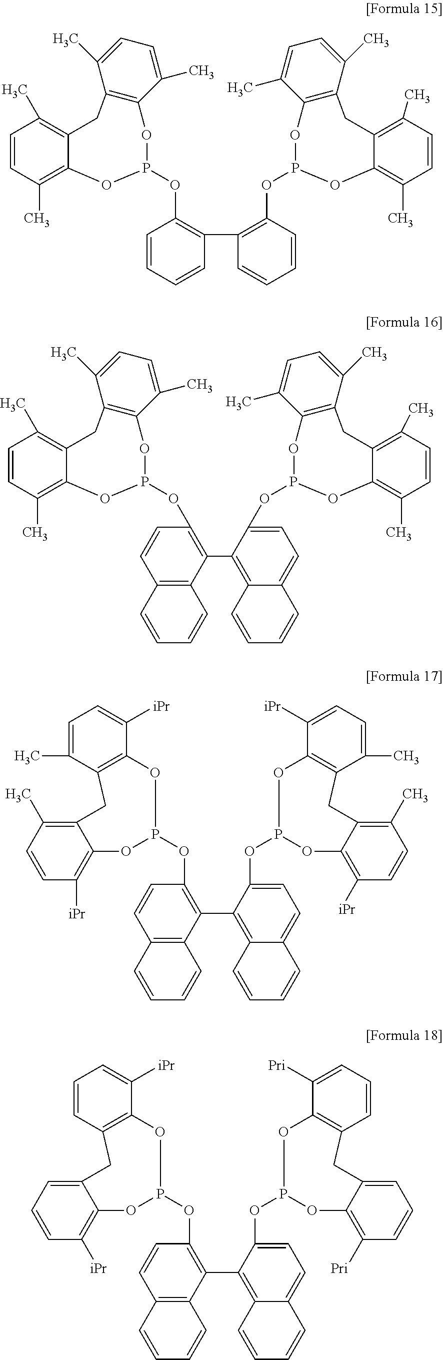

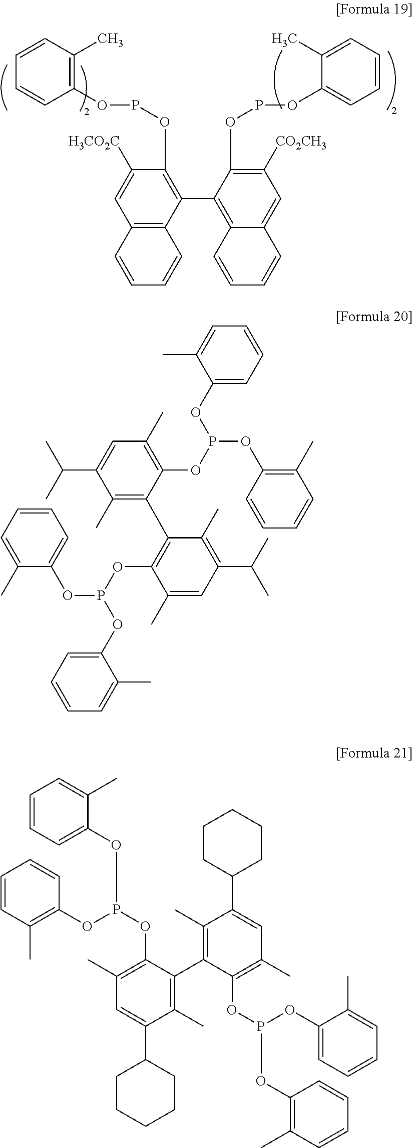

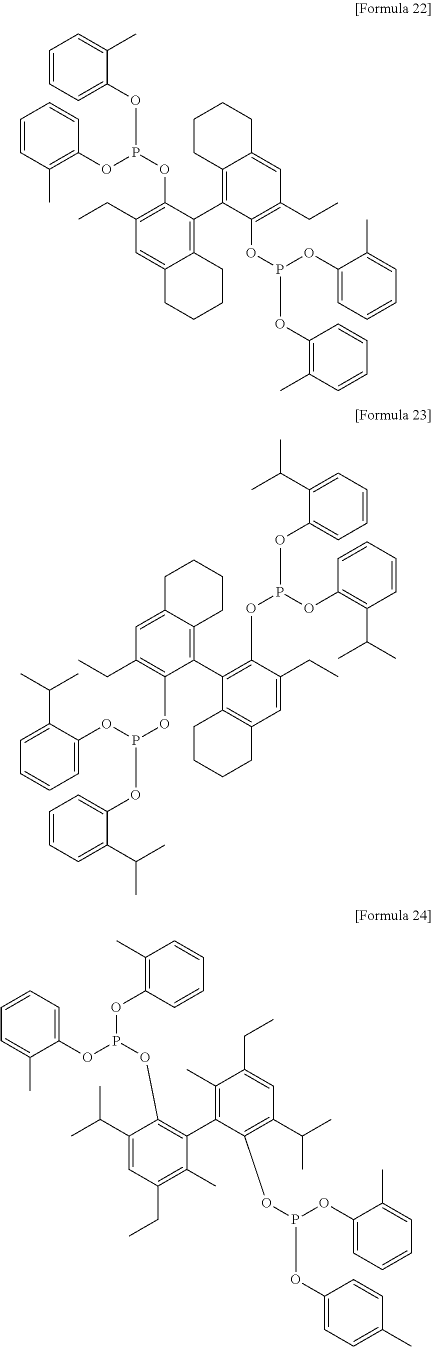 [SMILES: CCc1cc2c(c(-c3c4c(cc(CC)c3OP(Oc3ccccc3C)Oc3ccccc3C)CCCC4)c1OP(Oc1ccccc1C)Oc1ccccc1C)CCCC2 ; CCc1cc2c(c(-c3c4c(cc(CC)c3OP(Oc3ccccc3C(C)C)Oc3ccccc3C(C)C)CCCC4)c1OP(Oc1ccccc1C(C)C)Oc1ccccc1C(C)C)CCCC2 ; CCc1cc(C(C)C)c(OP(Oc2ccc(C)cc2)Oc2ccccc2C)c(-c2c(C)c(CC)cc(C(C)C)c2OP(Oc2ccccc2C)Oc2ccccc2C)c1C]

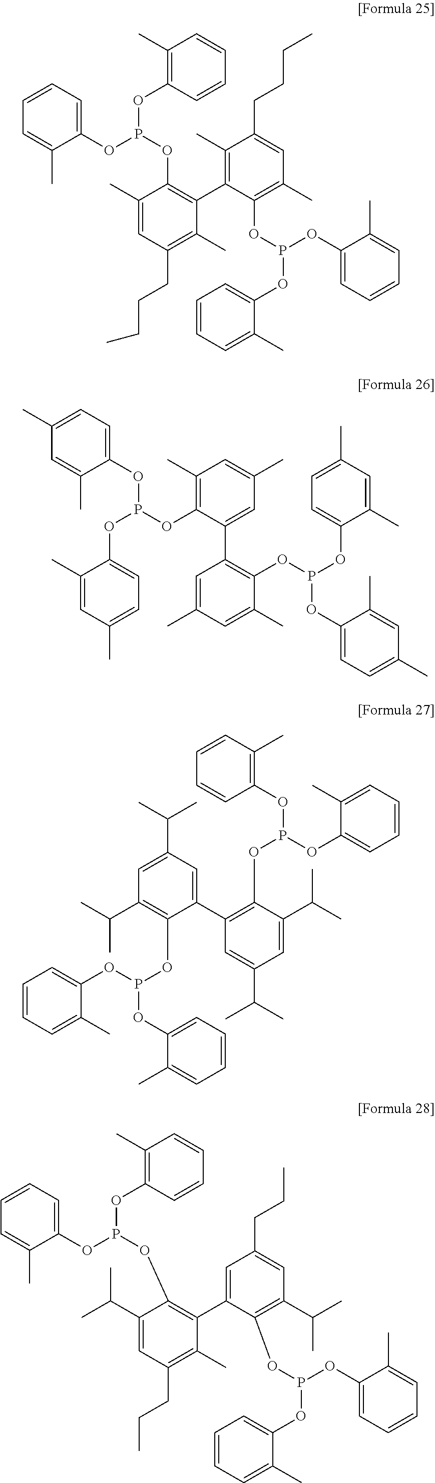

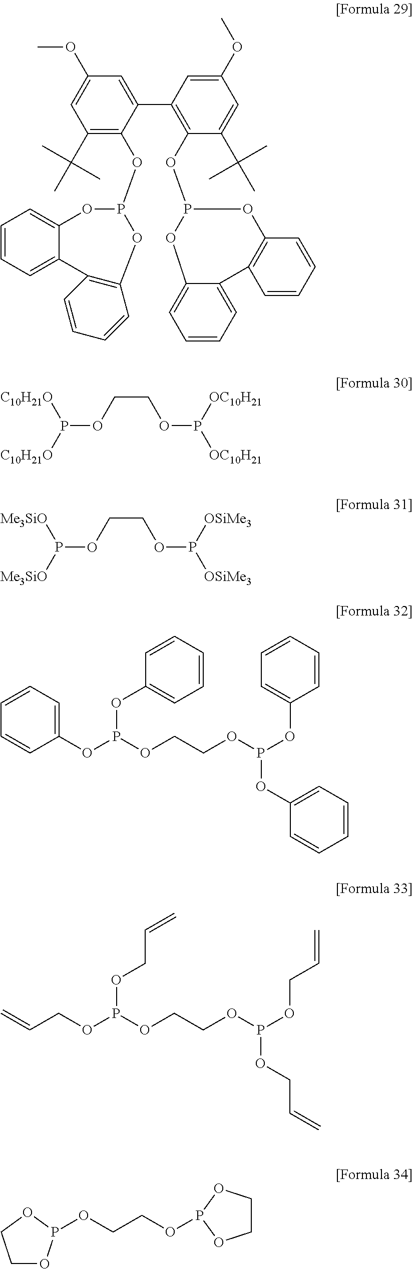

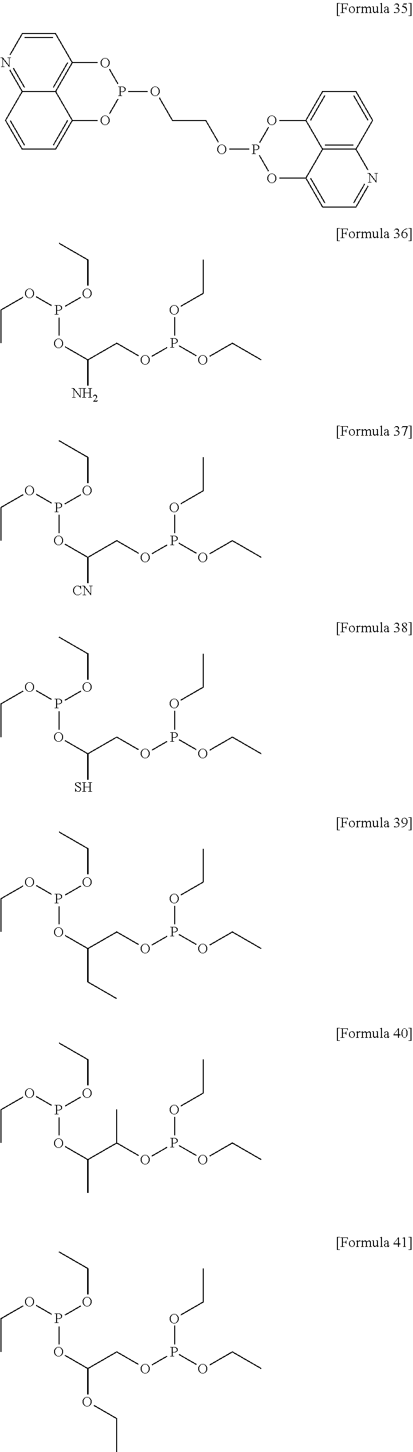

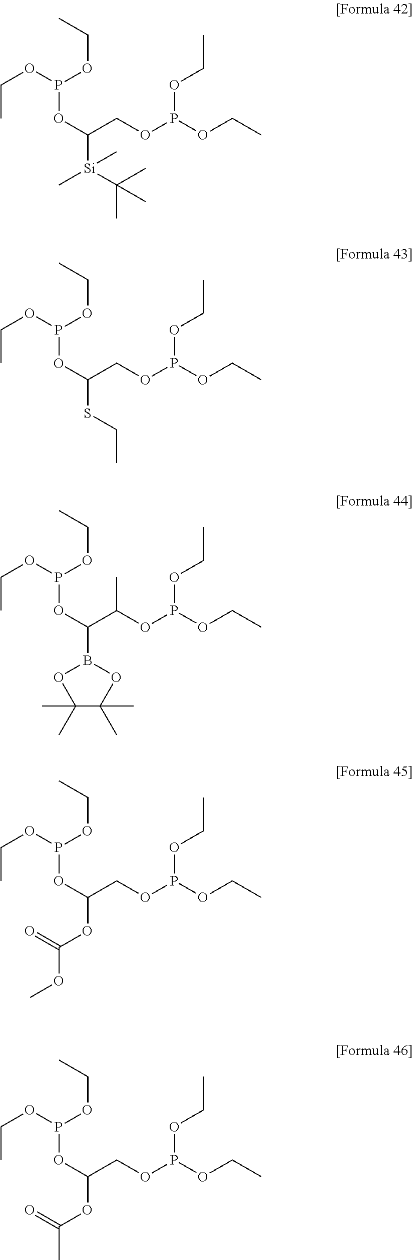

The compound represented by Chemical Formula 1 may include at least one of 6,6′-[(3,3′-di-t-butyl)-5,5′-dimethoxy-1,1′-biphenyl-2,2′-diyl)bis(oxy)]bis(dibenzo[d,f][1,3,2]dioxaphosphepin)hemi ethyl acetate) and 1,4-bis(1,3,2-dioxaphosphacyclopentyl)-1,4-dioxabutane.

The embodiments may also be realized by providing a lithium secondary battery including a positive electrode including a positive active material layer capable of intercalating and deintercalating lithium; a negative electrode including a negative active material layer; a separator between the positive electrode and negative electrode; and the non-aqueous electrolyte according to an embodiment.

The positive active material may include one selected from LiₐA_{1-b}R_{b}D₂ (0.90≦a≦1.8 and 0≦b≦0.5), LiₐE_{1-b}R_{b}O_{2-c}D_{c} (0.90≦a≦1.8, 0≦b≦0.5 and 0≦c≦0.05), LiE_{2-b}R_{b}O_{4-c}D_{c} (0≦b≦0.5, 0≦c≦0.05), LiₐNi_{1-b-c}Co_{b}R_{c}D_{α} (0.90≦a≦1.8, 0≦b≦0.5, 0≦c≦0.05 and 0≦α≦2), LiₐNi_{1-b-c}Co_{b}R_{c}O_{2-α}Z_{α} (0.90≦a≦1.8, 0≦b≦0.5, 0≦c≦0.05 and 0≦α≦2), LiₐNi_{1-b-c}Co_{b}R_{c}O_{2-α}Z_{α} (0.90≦a≦1.8, 0≦b≦0.5, 0≦c≦0.05 and 0≦α≦2), LiₐNi_{1-b-c}Mn_{b}R_{c}D_{α} (0.90≦a≦1.8, 0≦b≦0.5, 0≦c≦0.05 and 0≦α≦2), LiₐNi_{1-b-c}Mn_{b}R_{c}O_{2-α}Z_{α} (0.90≦a≦1.8, 0≦b≦0.5, 0≦c≦0.05 and 0≦α≦2), LiₐNi_{1-b-c}Mn_{b}R_{c}O_{2-α}Z₂ (0.90≦a≦1.8, 0≦b≦0.5, 0≦c≦0.05 and 0≦α≦2), LiₐNi_{b}E_{c}G_{d}O₂ (0.90≦a≦1.8, 0≦b≦0.9, 0≦c≦0.5 and 0.001≦d≦0.1), LiₐNi_{b}Co_{c}Mn_{d}GeO₂ (0.90≦a≦1.8, 0≦b≦0.9, 0≦c≦0.5, 0≦d≦0.5 and 0.001≦e≦0.1), LiₐNi_{b}G_{b}O₂ (0.90≦a≦1.8 and 0.001≦b≦0.1), LiₐCoG_{b}O₂ (0.90≦a≦1.8 and 0.001≦b≦0.1), LiₐMnG_{b}O₂ (0.90≦a≦1.8 and 0.001≦b≦0.1), LiₐMn₂G_{b}O₄ (0.90≦a≦1.8 and 0.001≦b≦0.1), QO₂; QS₂; LiQS₂; V₂O₅; LiV₂O₅; LiTO₂; LiNiVO₄; Li_{(3-f)}J₂(PO₄)₃ (0≦f≦2); Li_{(3-f)}Fe₂(PO₄)₃ (0≦f≦2), LiFePO₄, and a combination thereof, wherein, in the above formulae, A is Ni, Co, Mn, or a combination thereof; R is Al, Ni, Co, Mn, Cr, Fe, Mg, Sr, V, a rare earth element, or a combination thereof, D is O, F, S, P, or a combination thereof; E is Co, Mn, or a combination thereof, Z is F, S, P, or a combination thereof, G is Al, Cr, Mn, Fe, Mg, La, Ce, Sr, V, or a combination thereof, Q is Ti, Mo, Mn, or a combination thereof, T is Cr, V, Fe, Sc, Y, or a combination thereof, and J is V, Cr, Mn, Co, Ni, Cu, or a combination thereof.

The embodiments may also be realized by providing a lithium secondary battery including a positive electrode including a positive active material layer capable of intercalating and deintercalating lithium; a negative electrode including a negative active material layer capable of intercalating and deintercalating lithium; a separator between the positive electrode and negative electrode; and an electrolyte including a bidentate phosphorus compound represented by the following Chemical Formula 1, the compound represented by Chemical Formula 1 linking to a surface of the positive electrode or forming a chelate with a metal ion eluted from the positive active material,

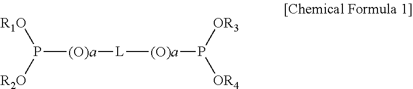

wherein a is independently 0 or 1, and R₁, R₂, R₃, and R₄ are each independently selected from the group of a substituted or unsubstituted C1 to C15 alkyl group, a substituted or unsubstituted C3 to C15 alkylsilyl group, a substituted or unsubstituted C3 to C15 cycloalkyl group, a substituted or unsubstituted C3 to C15 heterocycloalkyl group, a substituted or unsubstituted C6 to C15 aryl group, a substituted or unsubstituted C2 to C15 heteroaryl group, and a substituted or unsubstituted C2 to C15 alkenyl group, or R₁, R₂, R₃ and R₄ are independently linked together to provide one selected from the group of an oxygen-containing substituted or unsubstituted C2 to C15 heterocycloalkyl group and an oxygen-containing substituted or unsubstituted C2 to C15 heteroaryl group, and L is selected from the group of a C2 to C15 alkenylene group, a covalent bond bridging group represented by the following Chemical Formula 2, and a covalent bond bridging group represented by the following Chemical Formula 3,

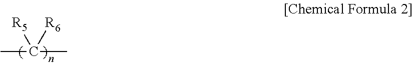

wherein n is an integer of 1 to about 4, and R₅ and R₆ are linked together to provide one selected from the group of a substituted or unsubstituted C3 to C15 cycloalkyl group, a substituted or unsubstituted C3 to C15 heterocycloalkyl group, a substituted or unsubstituted C3 to C20 aryl group, and a substituted or unsubstituted C2 to C20 heteroaryl group, or R₅ and R₆ are each independently selected from the group of hydrogen, an amine group, a cyano group, a thiolate group, a substituted or unsubstituted C1 to C15 alkyl group, —OR₇, —SiR₈R₉R₁₀, —OSiR₁₁R₁₂R₁₃, —SR₁₄, —SOR₁₅, —BR₁₆R₁₇, —OBR₁₈R₁₉, —OCOR₂₀ and —COR₂₁, in which R₇ to R₁₉ are each independently a substituted or unsubstituted C1 to C15 alkyl group, a substituted or unsubstituted C3 to C15 cycloalkyl group, a substituted or unsubstituted C3 to C15 heterocycloalkyl group, a substituted or unsubstituted C6 to C15 aryl group, or a substituted or unsubstituted C2 to C15 heteroaryl group,

(CH₂)_{b}-(A,X)Ar(Y,D)-(CH₂)_{b} [Chemical Formula 3]

wherein b is independently 0 to about 6, Ar is a substituted or unsubstituted C6 to C30 aryl group or a substituted or unsubstituted C1 to C30 heteroaryl group, and A, X, Y, and D are a substituent of Ar, and are each independently selected from the group of hydrogen, a substituted or unsubstituted C1 to C15 alkyl group, a substituted or unsubstituted C3 to C15 cycloalkyl group, a substituted or unsubstituted C3 to C15 heterocycloalkyl group, a substituted or unsubstituted C6 to C20 aryl group, a substituted or unsubstituted C2 to C20 heteroaryl group, a substituted or unsubstituted C2 to C15 alkenyl group, a substituted or unsubstituted C2 to C15 alkynyl group, a halo group, a cyano group, a nitro group, —OH, —OR₂₀, —COR₂₁, —COOR₂₂, —SH, —SR₂₃, —COSR₂₄, —NH₂, —NHR₂₅, —NR₂₆R₂₇, —NO₂, —CN, —CONH₂, —CONHR₂₈, and —CONR₂₉R₃₀, in which R₂₀ to R₃₀ are each independently a C1 to C15 alkyl group.

The chelate may be a cyclic chelate.

The compound represented by Chemical Formula 1 may be included in the electrolyte in an amount of about 0.01 wt % to about 20 wt %, based on a total weight of the electrolyte.

The compound represented by Chemical Formula 1 may include at least one of 1,3-bis(diphenoxyphosphine)propane, 1,3-bis(di(trimethylsiloxy)phosphine)methane, bis[1,3,2-dioxaphospholanyl-2]methane, 1,3-bis-[1,3,2-dioxaphospholanyl-2]propane, 1,2-bis[di(t-butoxy)phosphinomethyl]benzene, 1,2-bis[di(trimethylsiloxy)phosphinomethyl]benzene, 1,2-bis[di(t-butoxy)phosphinomethyl]-3,6-diphenyl-4,5-dimethylbenzene, 1,2-bis[di(t-butoxy)phosphinomethyl]-4,5-diphenylbenzene, 1,2-bis(decyloxy phosphorous acid)ethane, 1,4-bis(di(trimethylsiloxy)phosphino)-1,4-dioxabutane, phosphorous acid 2-(diphenoxy-phosphanyloxy)-ethyl ester diphenyl ester, phosphorous acid diallyl ester 2-(bis-allyloxy-phosphanyloxy)-ethyl ester, 1,4-bis(1,3,2-dioxaphosphacyclopentyl)-1,4-dioxabutane), 2-[2-(1,3-dioxa-6-aza-2-phospha-phenalen-2-yloxy)-ethoxy]-1,3-dioxa-6-aza-2-phospha-phenalene), phosphorous acid 2-amino-2-(diethoxy-phosphanyloxy)-ethyl ester diethyl ester, phosphorous acid 2-cyano-2-(diethoxy-phosphanyloxy)-ethyl ester diethyl ester, phosphorous acid 2-(diethoxy-phosphanyloxy)-2-mercapto-ethyl ester diethyl ester, phosphorous acid 2-(diethoxy-phosphanyloxy)-butyl ester diethyl ester, phosphorous acid 2-(diethoxy-phosphanyloxy)-1-methyl-propyl ester diethyl ester, phosphorous acid 2-(diethoxy-phosphanyloxy)-2-ethoxy-ethyl ester diethyl ester, phosphorous acid 2-(tert-butyl-dimethyl-silanyl)-2-(diethoxy-phosphanyloxy)-ethyl ester diethyl ester, phosphorous acid 2-(diethoxy-phosphanyloxy)-2-trimethylsilanyloxy-ethyl ester diethyl ester, phosphorous acid 2-(diethoxy-phosphanyloxy)-2-ethylsulfanyl-ethyl ester diethyl ester, phosphorous acid 2-(diethoxy-phosphanyloxy)-2-(4,4,5,5-tetramethyl-[1,3,2]dioxaborolan-2-yl)-ethyl ester diethyl ester, carbonic acid 1,2-bis-(diethoxy-phosphanyloxy)-ethyl ester methyl ester, acetic acid 1,2-bis-(diethoxy-phosphanyloxy)-ethyl ester, and compounds represented by the following Chemical Formulas 11 to 46, in which iPr denotes isopropyl,

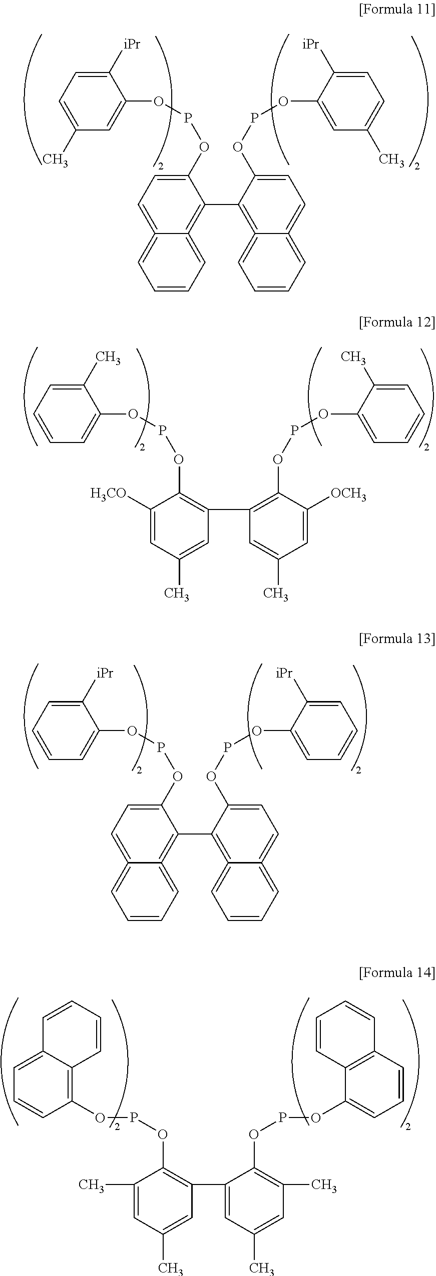

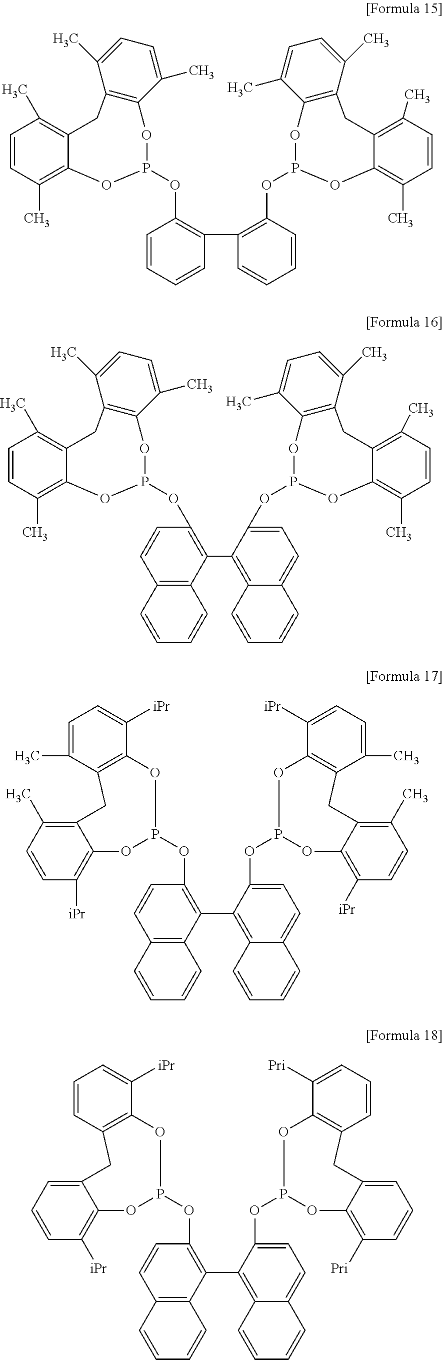

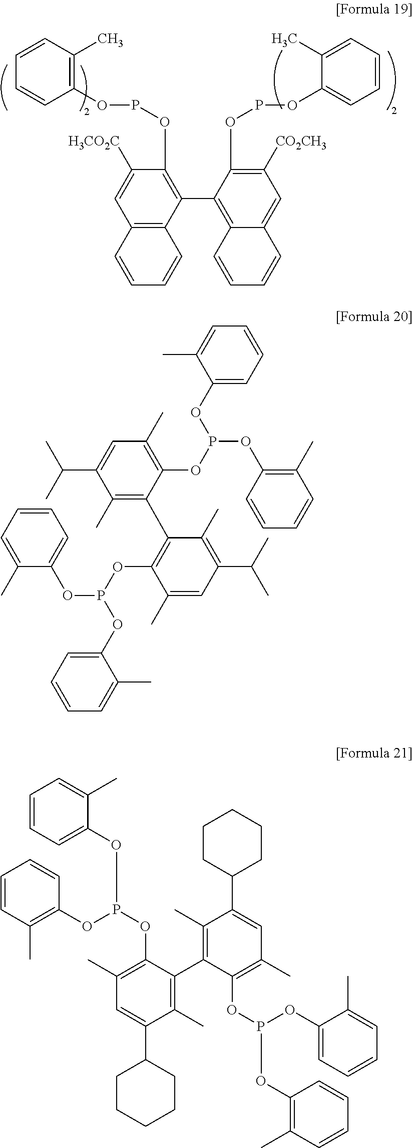

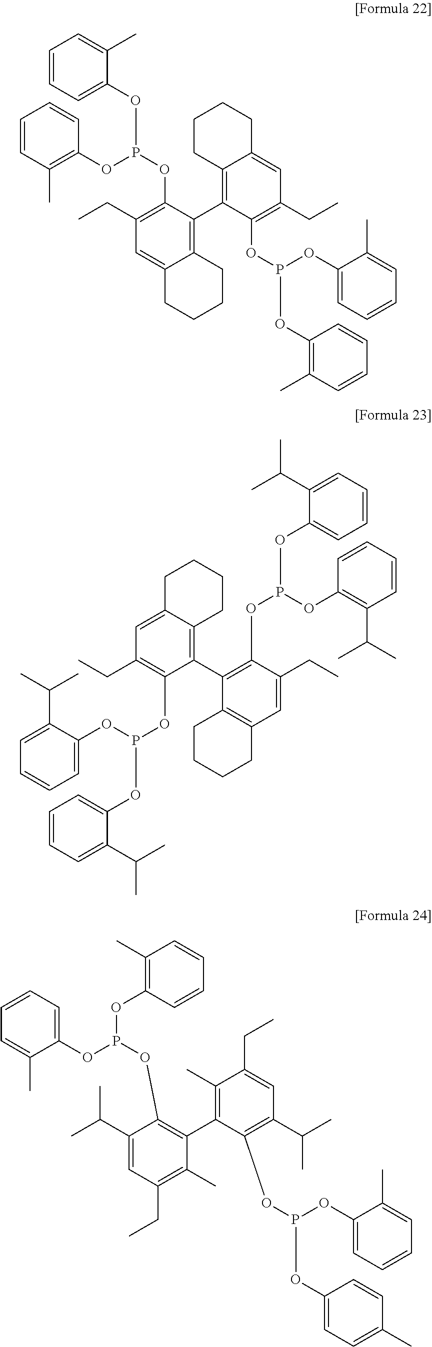 [SMILES: CCc1cc2c(c(-c3c4c(cc(CC)c3OP(Oc3ccccc3C)Oc3ccccc3C)CCCC4)c1OP(Oc1ccccc1C)Oc1ccccc1C)CCCC2 ; CCc1cc2c(c(-c3c4c(cc(CC)c3OP(Oc3ccccc3C(C)C)Oc3ccccc3C(C)C)CCCC4)c1OP(Oc1ccccc1C(C)C)Oc1ccccc1C(C)C)CCCC2 ; CCc1cc(C(C)C)c(OP(Oc2ccc(C)cc2)Oc2ccccc2C)c(-c2c(C)c(CC)cc(C(C)C)c2OP(Oc2ccccc2C)Oc2ccccc2C)c1C]

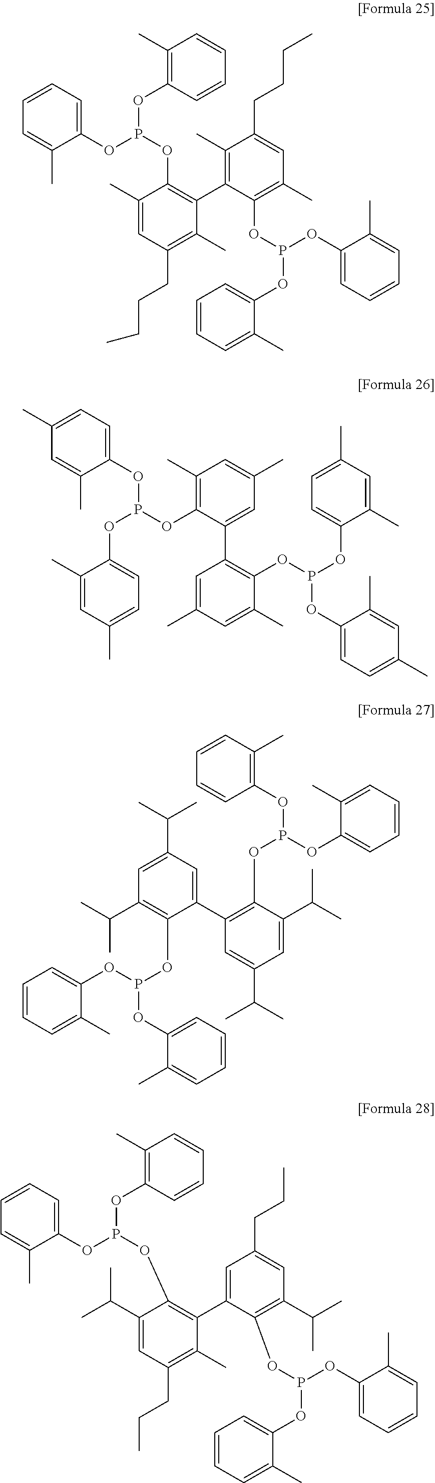

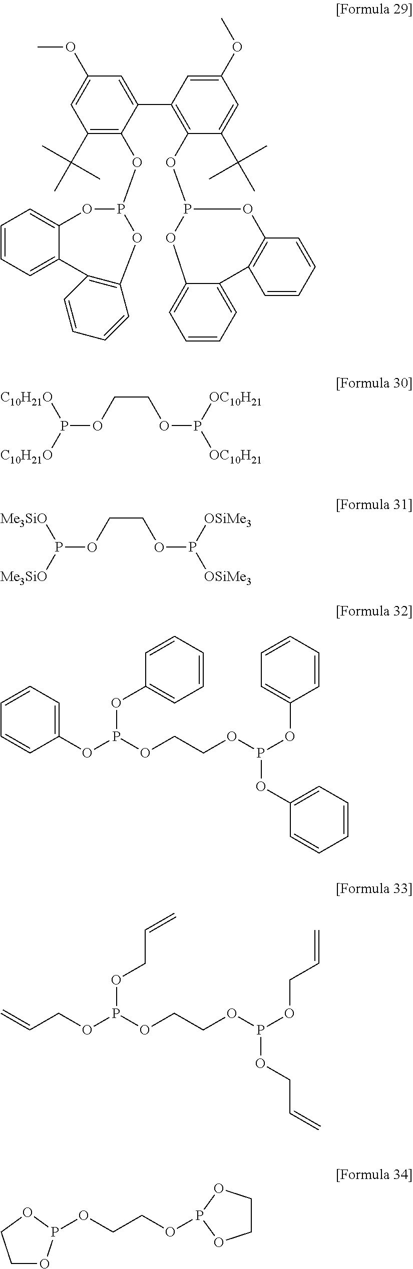

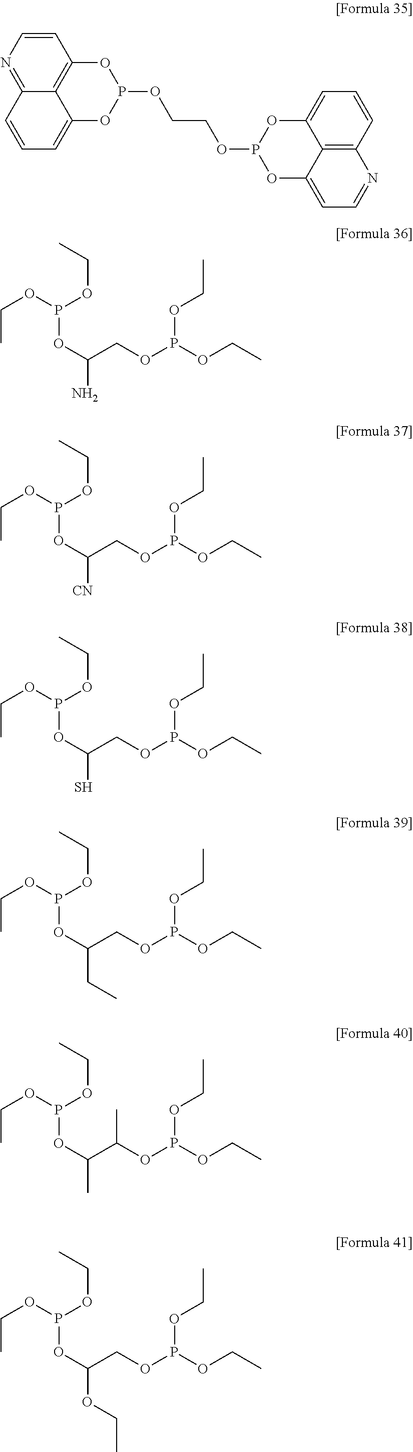

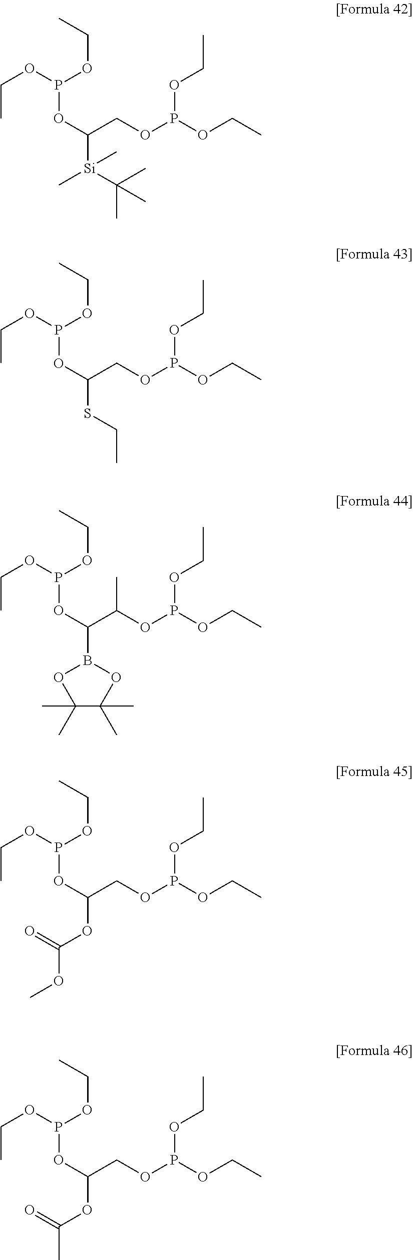

The compound represented by Chemical Formula 1 may include at least one of 6,6′-[(3,3′-di-t-butyl)-5,5′-dimethoxy-1,1′-biphenyl-2,2′-diyl)bis(oxy)]bis(dibenzo[d,f][1,3,2]dioxaphosphepin)hemi ethyl acetate) and 1,4-bis(1,3,2-dioxaphosphacyclopentyl)-1,4-dioxabutane.

### BRIEF DESCRIPTION OF THE DRAWINGS

The embodiments will become apparent to those of ordinary skill in the art by describing in detail exemplary embodiments with reference to the attached drawings, in which:

FIG. 1 illustrates a schematic view of a lithium secondary battery according to an embodiment.

FIG. 2 illustrates a graph showing results of internal resistance and capacitance retention of Experimental Examples 1 and 2 and Comparative Experimental Examples 1 and 2 after 90 cycles.

### DETAILED DESCRIPTION

This application claims priority to and the benefit of Korean Patent Application No. 10-2011-0065111 filed in the Korean Intellectual Property Office on Jun. 30, 2011, the entire contents of which are incorporated herein by reference.

Example embodiments will now be described more fully hereinafter with reference to the accompanying drawings; however, they may be embodied in different forms and should not be construed as limited to the embodiments set forth herein. Rather, these embodiments are provided so that this disclosure will be thorough and complete, and will fully convey the scope of the invention to those skilled in the art.

In the drawing figures, the dimensions of layers and regions may be exaggerated for clarity of illustration. It will also be understood that when a layer or element is referred to as being “on” another element, it can be directly on the other element, or intervening elements may also be present. In addition, it will also be understood that when an element is referred to as being “between” two elements, it can be the only element between the two elements, or one or more intervening elements may also be present. Like reference numerals refer to like elements throughout.

Well-known technologies may not be specifically illustrated to avoid obscuring aspects of the embodiments. Unless another definition is provided, all the terms mentioned in the specification (including technological and scientific terms) are easily understood to those who have common knowledge in a field related to the present invention. In addition, unless explicitly described to the contrary, the word “comprise,” and variations such as “comprises” or “comprising,” will be understood to imply the inclusion of stated elements but not the exclusion of any other elements. Furthermore, a singular form covers a plural form, unless specifically mentioned otherwise.

As used herein, when a definition is not otherwise provided, the term “substituted” may refer to one substituted with a substituent selected from the group of a C1 to C15 alkyl group, a C1 to C15 alkoxy group, a carboxyl group, a C2 to C15 alkenyl group, a C2 to C15 alkynyl group, a C3 to C15 cycloalkyl group, a C3 to C15 cycloalkenyl group, a C3 to C15 cycloalkynyl group, a C3 to C15 heterocycloalkyl group, a C3 to C15 heterocycloalkenyl group, a C3 to C15 heterocycloalkynyl group, a C6 to C20 aryl group, and a C2 to C20 heteroaryl group.

As used herein, when a definition is not otherwise provided, the prefix “hetero” may refer to a functional group including 1 to 3 heteroatoms selected from the group of N, O, S, P, and Si.

The non-aqueous electrolyte additive according to an embodiment may include a bidentate phosphorus compound, e.g., a bidentate phosphine compound or a bidentate alkoxyphosphine compound. The additive may include a bidentate phosphorus compound represented by the following Chemical Formula 1.

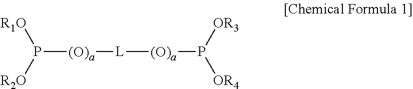

In Chemical Formula 1, a may be 0 or 1.

R₁, R₂, R₃, and R₄ may each independently be selected from the group of a substituted or unsubstituted C1 to C15 alkyl group, a substituted or unsubstituted C3 to C15 alkylsilyl group, a substituted or unsubstituted C6 to C15 aryl group, and a substituted or unsubstituted C2 to C15 alkenyl group.

Alternatively, R₁, R₂, R₃, and R₄ may be independently linked together to provide one selected from the group of an oxygen-containing substituted or unsubstituted C2 to C15 heterocycloalkyl group and an oxygen-containing substituted or unsubstituted C2 to C15 heteroaryl group. For example, R₁ may be linked with R₂ and/or R₃ may be linked with R₄.

L may be selected from the group of a C1 to C15 alkenyl group, a covalent bond bridging group represented by the following Chemical Formula 2, and a covalent bond bridging group represented by the following Chemical Formula 3.

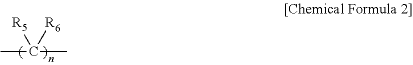

In Chemical Formula 2, n may be integer of 1 to about 4.

R₅ and R₆ may be linked together to provide one selected from the group of a substituted or unsubstituted C3 to C15 cycloalkyl group, a substituted or unsubstituted C3 to C15 heterocycloalkyl group, a substituted or unsubstituted C3 to C20 aryl group, and a substituted or unsubstituted C2 to C20 heteroaryl group.

Alternatively, R₅ and R₆ may each independently be selected from the group of hydrogen, an amine group, a cyano group, a thiolate group, a substituted or unsubstituted C1 to C15 alkyl group, an alkoxy group (—OR₇), a silyl group (—SiR₈R₉R₁₀), a siloxyl group (—OSiR₁₁R₁₂R₁₃), a sulfide group (—SR₁₄), a sulfoxide group (—SOR₁₅), a boryl group (—BR₁₆R₁₇), a borinic acid group (—OBR₁₈R₁₉), an ester group (—OCOR₂₀), and an acyl group (—COR₂₁).

R₇ to R₁₉ may each independently be selected from the group of a substituted or unsubstituted C1 to C15 alkyl group, a substituted or unsubstituted C3 to C15 cycloalkyl group, a substituted or unsubstituted C3 to C15 heterocycloalkyl group, a substituted or unsubstituted C6 to C20 aryl group, and a substituted or unsubstituted C2 to C20 heteroaryl group.

(CH₂)_{b}-(A,X)Ar(Y,D)-(CH₂)_{b} [Chemical Formula 3]

In Chemical Formula 3, b may be 0 to about 6.

Ar may be a substituted or unsubstituted C1 to C30 aryl group or a substituted or unsubstituted C1 to C30 heteroaryl group.

For example, A, X, Y, and D may be a substituent of Ar, and may each independently be selected from the group of hydrogen, a substituted or unsubstituted C1 to C15 alkyl group, a substituted or unsubstituted C3 to C15 cycloalkyl group, a substituted or unsubstituted C3 to C15 heterocycloalkyl group, a substituted or unsubstituted C6 to C20 aryl group, a substituted or unsubstituted C2 to C20 heteroaryl group, a substituted or unsubstituted C2 to C15 alkenyl group, a substituted or unsubstituted C2 to C15 alkynyl group, a halo group, a cyano group, a nitro group, —OH, —OR₂₀, —COR₂₁, —COOR₂₂, —SH, —SR₂₃, —COSR₂₄, —NH₂, —NHR₂₅, —NR₂₆R₂₇, —NO₂, —CN, —CONH₂, —CONHR₂₈, and —CONR₂₉R₃₀. R₂₀ to R₃₀ may each independently be a C1 to C15 alkyl group.

Non-limiting examples of the compound represented by Chemical Formula 1 may include 1,3-bis(diphenoxyphosphine)propane, 1,3-bis(di(trimethylsiloxy)phosphine)methane, bis[1,3,2-dioxaphospholanyl-2]methane, 1,3-bis-[1,3,2-dioxaphospholanyl-2]propane, 1,2-bis[di(t-butoxy)phosphinomethyl]benzene, 1,2-bis[di(trimethylsiloxy)phosphinomethyl]benzene, 1,2-bis[di(t-butoxy)phosphinomethyl]-3,6-diphenyl-4,5-dimethylbenzene, 1,2-bis[di(t-butoxy)phosphinomethyl]-4,5-diphenylbenzene, 1,2-bis(decyloxy phosphorous acid)ethane, 1,4-bis(di(trimethylsiloxy)phosphino)-1,4-dioxabutane, phosphorous acid 2-(diphenoxy-phosphanyloxy)-ethyl ester diphenyl ester, phosphorous acid diallyl ester 2-(bis-allyloxy-phosphanyloxy)-ethyl ester, 1,4-bis(1,3,2-dioxaphosphacyclopentyl)-1,4-dioxabutane), 2-[2-(1,3-dioxa-6-aza-2-phospha-phenalen-2-yloxy)-ethoxy]-1,3-dioxa-6-aza-2-phospha-phenalene), phosphorous acid 2-amino-2-(diethoxy-phosphanyloxy)-ethyl ester diethyl ester, phosphorous acid 2-cyano-2-(diethoxy-phosphanyloxy)-ethyl ester diethyl ester, phosphorous acid 2-(diethoxy-phosphanyloxy)-2-mercapto-ethyl ester diethyl ester, phosphorous acid 2-(diethoxy-phosphanyloxy)-butyl ester diethyl ester, phosphorous acid 2-(diethoxy-phosphanyloxy)-1-methyl-propyl ester diethyl ester, phosphorous acid 2-(diethoxy-phosphanyloxy)-2-ethoxy-ethyl ester diethyl ester, phosphorous acid 2-(tert-butyl-dimethyl-silanyl)-2-(diethoxy-phosphanyloxy)-ethyl ester diethyl ester, phosphorous acid 2-(diethoxy-phosphanyloxy)-2-trimethylsilanyloxy-ethyl ester diethyl ester, phosphorous acid 2-(diethoxy-phosphanyloxy)-2-ethylsulfanyl-ethyl ester diethyl ester, phosphorous acid 2-(diethoxy-phosphanyloxy)-2-(4,4,5,5-tetramethyl-[1,3,2]dioxaborolan-2-yl)-ethyl ester diethyl ester, carbonic acid 1,2-bis-(diethoxy-phosphanyloxy)-ethyl ester methyl ester, acetic acid 1,2-bis-(diethoxy-phosphanyloxy)-ethyl ester, and compounds represented by the following Chemical Formulas 11 to 46 (hereinafter, in the Chemical Formulas 11 to 46, iPr denotes isopropyl and Me denotes methyl).

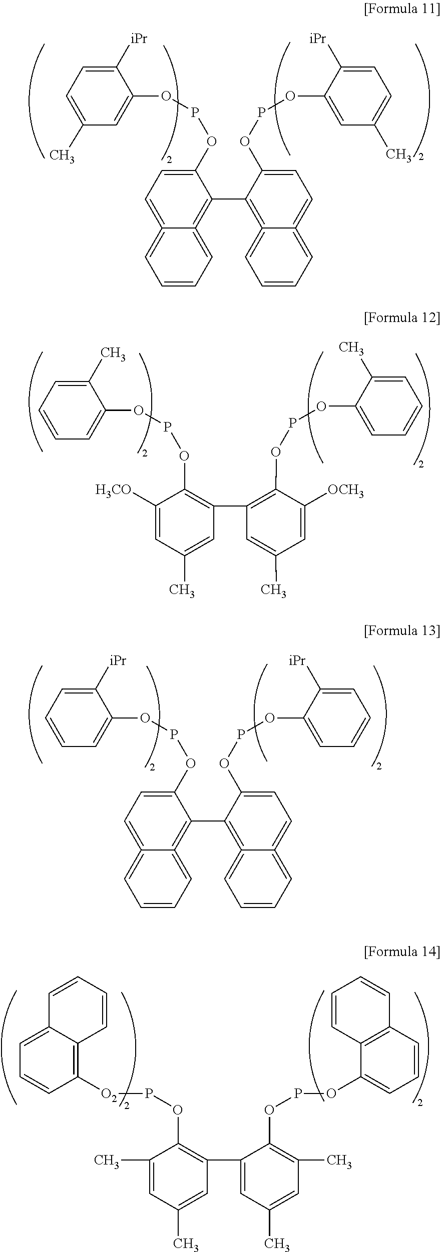

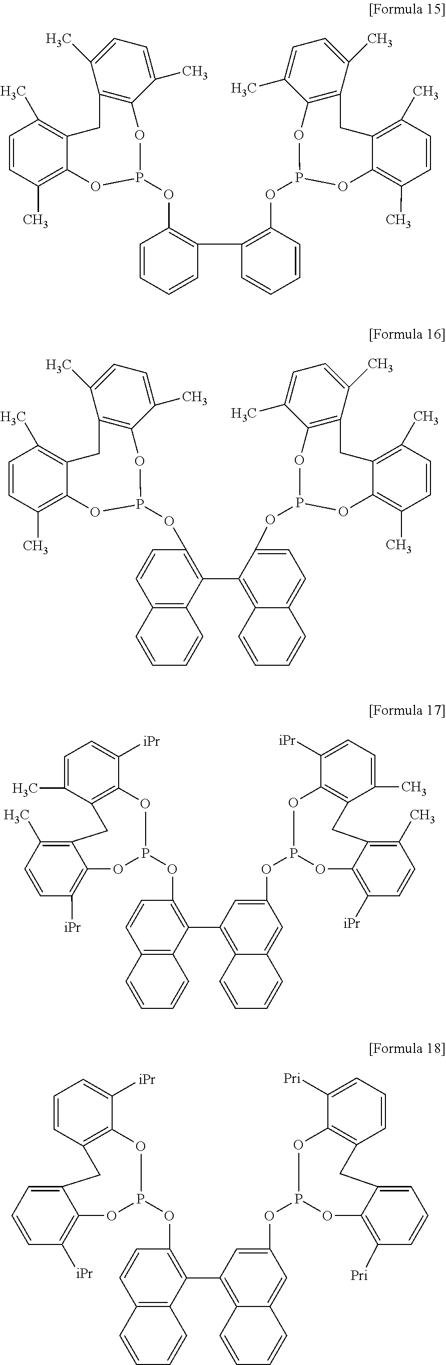

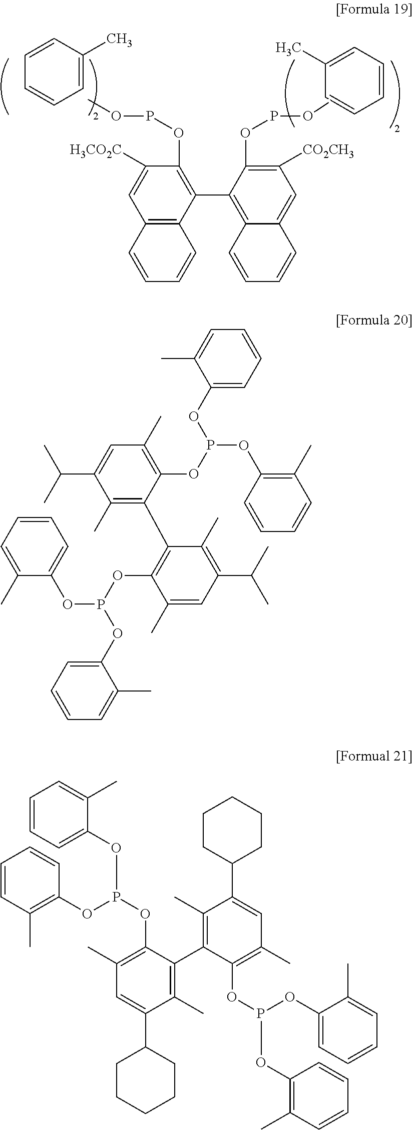

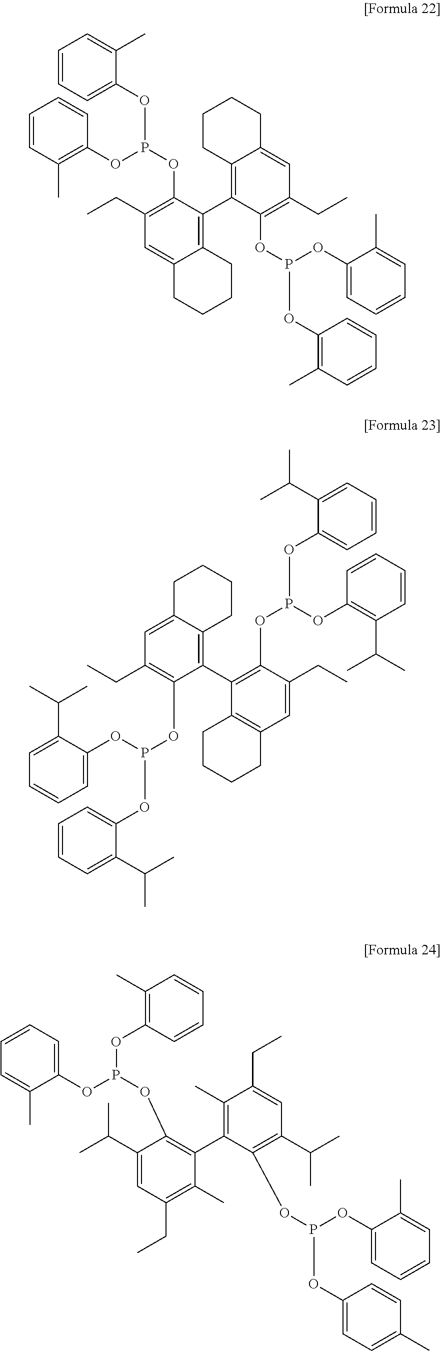 [SMILES: CCc1cc2c(c(-c3c4c(cc(CC)c3OP(Oc3ccccc3C)Oc3ccccc3C)CCCC4)c1OP(Oc1ccccc1C)Oc1ccccc1C)CCCC2 ; CCc1cc2c(c(-c3c4c(cc(CC)c3OP(Oc3ccccc3C(C)C)Oc3ccccc3C(C)C)CCCC4)c1OP(Oc1ccccc1C(C)C)Oc1ccccc1C(C)C)CCCC2 ; CCc1cc(C(C)C)c(OP(Oc2ccc(C)cc2)Oc2ccccc2C)c(-c2c(C)c(CC)cc(C(C)C)c2OP(Oc2ccccc2C)Oc2ccccc2C)c1C]

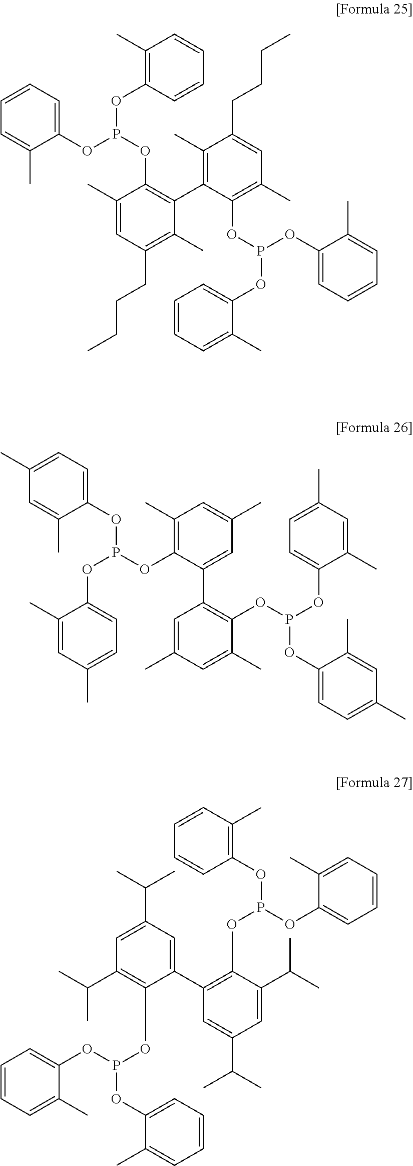

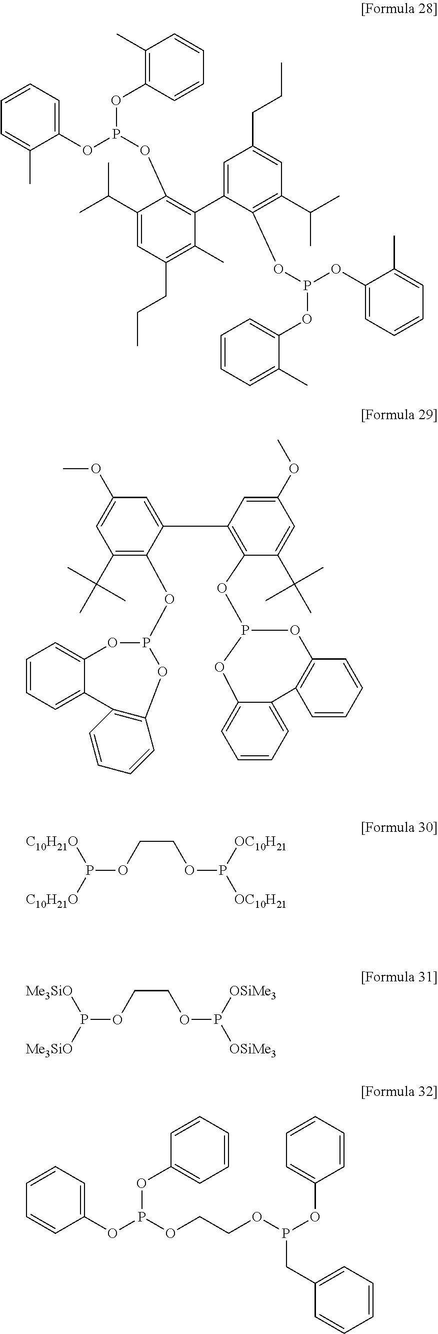

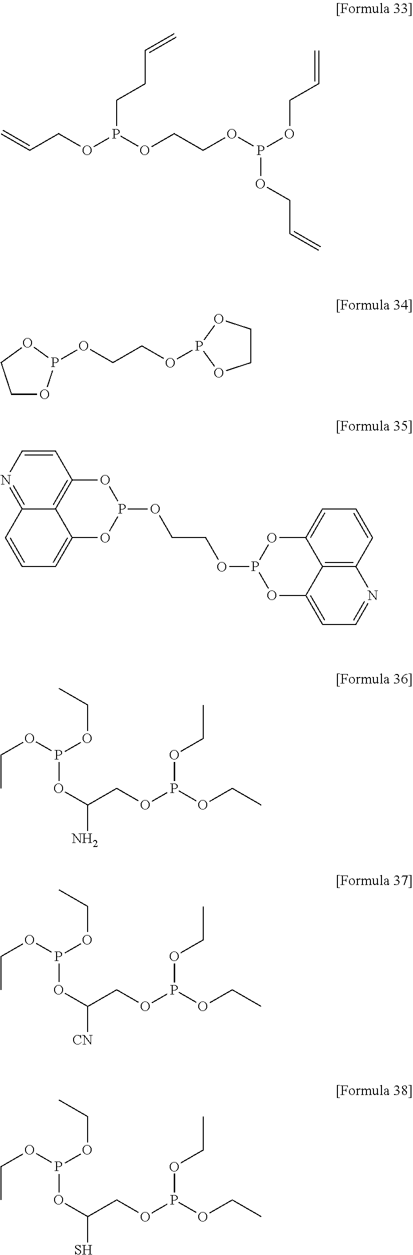

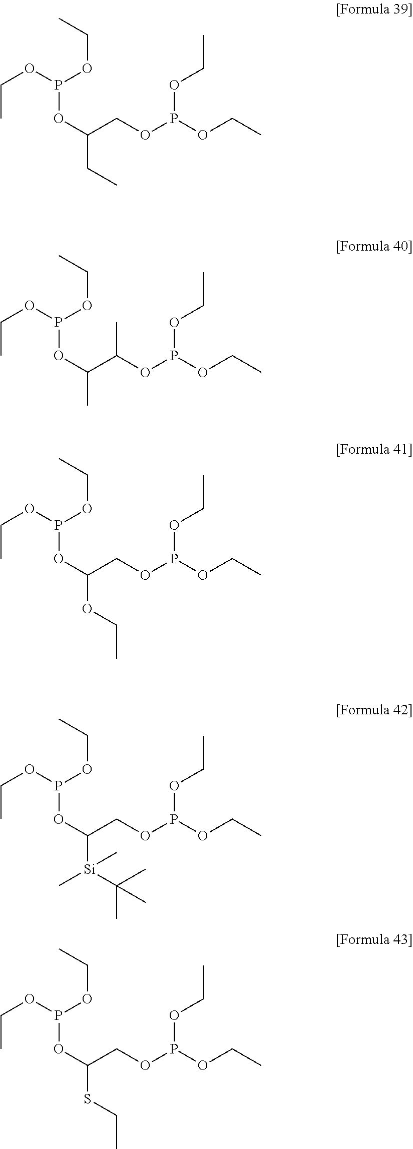 [SMILES: CCOP(OCC)OCC(CC)OP(OCC)OCC ; CCOP(OCC)OC(C)C(C)OP(OCC)OCC ; CCOC(COP(OCC)OCC)OP(OCC)OCC ; CCOP(OCC)OCC(OP(OCC)OCC)[Si](C)(C)C(C)(C)C ; CCOP(OCC)OCC(OP(OCC)OCC)SCC]

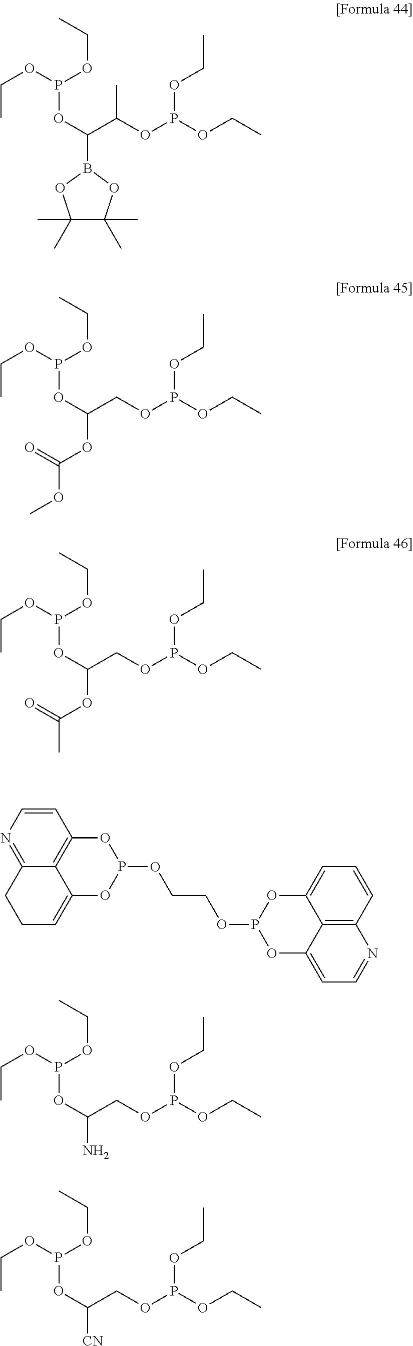 [SMILES: CCOP(OCC)OC(C)C(OP(OCC)OCC)B1OC(C)(C)C(C)(C)O1 ; CCOP(OCC)OCC(OC(=O)OC)OP(OCC)OCC ; CCOP(OCC)OCC(OC(C)=O)OP(OCC)OCC ; C1=C2OP(OCCOP3Oc4cccc5nccc(c45)O3)Oc3ccnc(c32)CC1 ; CCOP(OCC)OCC(N)OP(OCC)OCC ; CCOP(OCC)OCC(C#N)OP(OCC)OCC]

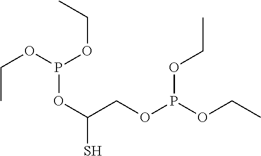 [SMILES: CCOP(OCC)OCC(S)OP(OCC)OCC]

The bidentate phosphorus compound for the additive may provide a stable coating layer on a surface of a positive active material layer and/or a stable cyclic chelate with a metal ion.

A non-aqueous electrolyte according to an embodiment may include a base electrolyte (including a non-aqueous organic solvent and a lithium salt) and the additive according to an embodiment.

The additive may include the bidentate phosphorus compound according to an embodiment. An amount of the additive included in the electrolyte may be adjusted to help improve performance of a lithium secondary battery. In an implementation, the additive may be included in an amount of about 0.01 wt % to about 20 wt %, e.g., about 0.01 wt % to about 10 wt %, based on a total weight of the base electrolyte. When the additive is included within the range, stability and high temperature stability of a positive electrode may be enhanced and a detrimental effect due to a side reaction of additive may be simultaneously minimized. Accordingly, an electrolyte exhibiting an appropriate conductivity and viscosity as well as excellent performance may be provided.

The non-aqueous organic solvent may function as a medium for transmitting or transporting ions taking part in an electrochemical reaction of a battery. The non-aqueous organic solvent may include, e.g., a carbonate-based, an ester-based, an ether-based, a ketone-based, an alcohol-based, and/or an aprotic solvent. Examples of the carbonate-based solvent may include dimethyl carbonate (DMC), diethyl carbonate (DEC), dipropyl carbonate (DPC), methylpropyl carbonate (MPC), ethylpropyl carbonate (EPC), methylethyl carbonate (MEC), ethylene carbonate (EC), propylene carbonate (PC), butylene carbonate (BC), and the like. Examples of the ester-based solvent may include methyl acetate, ethyl acetate, n-propyl acetate, dimethylacetate, methylpropionate, ethylpropionate, γ-butyrolactone, decanolide, valerolactone, mevalonolactone, caprolactone, and the like. Examples of the ether-based solvent may include dibutyl ether, tetraglyme, diglyme, dimethoxyethane, 2-methyltetrahydrofuran, tetrahydrofuran, and the like. Examples of the ketone-based solvent may include cyclohexanone and the like. Examples of the alcohol-based solvent may include ethyl alcohol, isopropyl alcohol, and the like. Examples of the aprotic solvent may include nitriles, R—CN(R being a C2 to C20 linear, branched, or cyclic hydrocarbon group including a double bond, an aromatic ring, or an ether bond), amides such as dimethyl formamide, dioxolanes such as 1,3-dioxolane, sulfolanes, and the like.

The non-aqueous organic solvent may be used singularly or in a mixture. When the organic solvent is used in a mixture, a mixture ratio may be controlled in accordance with a desired battery performance.

The carbonate-based solvent may include a mixture of a cyclic carbonate and a linear carbonate. The cyclic carbonate and the linear carbonate may be mixed together in the volume ratio of about 1:1 to about 1:9, and when such a mixture is used as an electrolyte, electrolyte performance may be enhanced.

In an implementation, the electrolyte may further include mixtures of carbonate-based solvents and aromatic hydrocarbon-based solvents. The carbonate-based solvents and the aromatic hydrocarbon-based solvents may be mixed together in a volume ratio of about 1:1 to about 30:1.

The aromatic hydrocarbon-based solvent may include a compound represented by Chemical Formula 4, below.

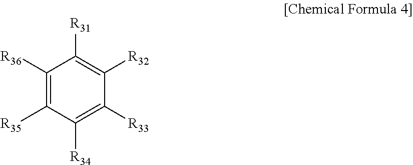

In Chemical Formula 4, R₃₁ to R₃₆ may each independently include hydrogen, a halogen, a C1 to C10 alkyl group, a C1 to C10 haloalkyl group, or a combination thereof.

The aromatic hydrocarbon-based organic solvent may include, e.g., benzene, fluorobenzene, 1,2-difluorobenzene, 1,3-difluorobenzene, 1,4-difluorobenzene, 1,2,3-trifluorobenzene, 1,2,4-trifluorobenzene, chlorobenzene, 1,2-dichlorobenzene, 1,3-dichlorobenzene, 1,4-dichlorobenzene, 1,2,3-trichlorobenzene, 1,2,4-trichlorobenzene, iodobenzene, 1,2-diiodobenzene, 1,3-diiodobenzene, 1,4-diiodobenzene, 1,2,3-triiodobenzene, 1,2,4-triiodobenzene, toluene, fluorotoluene, 1,2-difluorotoluene, 1,3-difluorotoluene, 1,4-difluorotoluene, 1,2,3-trifluorotoluene, 1,2,4-trifluorotoluene, chlorotoluene, 1,2-dichlorotoluene, 1,3-dichlorotoluene, 1,4-dichlorotoluene, 1,2,3-trichlorotoluene, 1,2,4-trichlorotoluene, iodotoluene, 1,2-diiodotoluene, 1,3-diiodotoluene, 1,4-diiodotoluene, 1,2,3-triiodotoluene, 1,2,4-triiodotoluene, xylene, or a combination thereof.

The non-aqueous electrolyte may further include vinylene carbonate or an ethylene carbonate-based compound represented by the following Chemical Formula 5, in order to improve cycle-life of a battery.

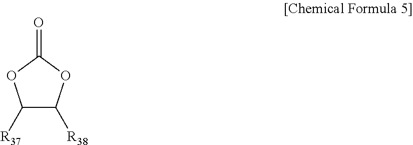

In Chemical Formula 5, R₃₇ and R₃₈ may each independently include hydrogen, a halogen, a cyano group (CN), a nitro group (NO₂) or a C1 to C5 fluoroalkyl group, provided that at least one of R₃₇ and R₃₈ is a halogen, a cyano group (CN), a nitro group (NO₂), or a C1 to C5 fluoroalkyl group.

Examples of the ethylene carbonate-based compound may include difluoro ethylenecarbonate, chloroethylene carbonate, dichloroethylene carbonate, bromoethylene carbonate, dibromoethylene carbonate, nitroethylene carbonate, cyanoethylene carbonate, fluoroethylene carbonate, and the like. An amount of the vinylene carbonate or the ethylene carbonate-based compound included in the electrolyte may be adjusted within an appropriate range in order to improve cycle life.

Non-limiting examples of the lithium salt may include at least one supporting salt selected from LiPF₆, LiBF₄, LiSbF₆, LiAsF₆, LiC₄F₉SO₃, LiClO₄, LiAlO₂, LiAlCl₄, LiN(CₓF₂ₓ₊₁SO₂)(C_{y}F_{2y+1}SO₂), (where x and y are natural numbers), LiCl, LiI, LiB(C₂O₄)₂ (lithium bis(oxalato)borate, LiBOB), or a combination thereof. The lithium salt may be used at a concentration of about 0.1 to about 2.0 M.

When the lithium salt is included at the above concentration range, electrolyte performance and lithium ion mobility may be enhanced due to optimal electrolyte conductivity and viscosity.

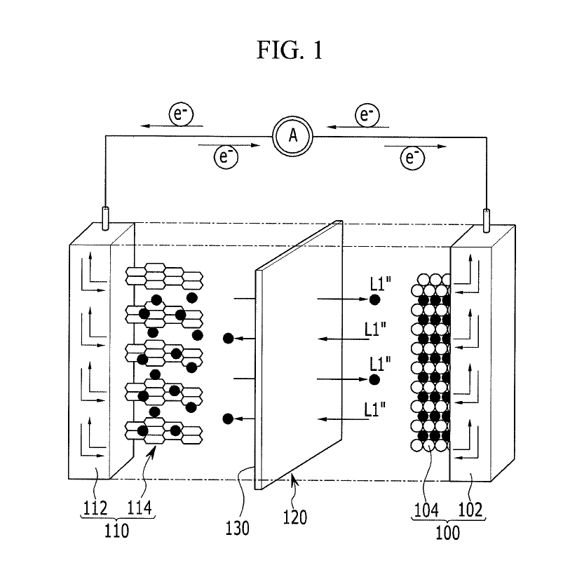

Hereinafter, referring to FIG. 1, a lithium secondary battery including the non-aqueous electrolyte according to an embodiment is described. FIG. 1 illustrates a schematic view of a lithium secondary battery according to an embodiment. Referring to FIG. 1, the lithium secondary battery according to the present embodiment may include a positive electrode 100, a negative electrode 110, a separator 120 between the positive electrode 100 and negative electrode 110, and a non-aqueous electrolyte 130 impregnated in or surrounding the positive electrode 100, negative electrode 110, and separator 120.

The positive electrode 100 may include a current collector 102 and a positive active material layer 104 on the current collector 102.

The current collector 102 may include any suitable metal having high conductivity, being easily attached to the positive active material layer 104 lithium secondary battery, and having little to no reactivity within a voltage range of a lithium secondary battery. For example, the current collector 102 may be formed of an aluminum (Al) thin film or an aluminum alloy thin film, but is not limited thereto.

A positive active material of the positive active material layer 104 may include lithiated intercalation compounds that reversibly intercalate and deintercalate lithium ions. The positive active material may include a composite oxide including lithium and at least one selected from the group of cobalt, manganese, and nickel. Examples thereof may include, e.g., LiₐA_{1-b}R_{b}D₂ (0.90≦a≦1.8 and 0≦b≦0.5), LiₐE_{1-b}R_{b}O_{2-c}D_{c} (0.90≦a≦1.8, 0≦b≦0.5 and 0≦c≦0.05), LiE_{2-b}R_{b}O_{4-c}D_{c} (0≦b≦0.5, 0≦c≦0.05), LiₐNi_{1-b-c}Co_{b}R_{c}D_{α} (0.90≦a≦1.8, 0≦b≦0.5, 0≦c≦0.05 and 0≦α≦2), LiₐNi_{1-b-c}Co_{b}R_{c}O_{2-α}Z_{α} (0.90≦a≦1.8, 0≦b≦0.5, 0≦c≦0.05 and 0≦α≦2), LiₐNi_{1-b-c}Co_{b}R_{c}O_{2-α}Z₂ (0.90≦a≦1.8, 0≦b≦0.5, 0≦c≦0.05 and 0≦α≦2), LiₐNi_{1-b-c}Mn_{b}R_{c}D_{α} (0.90≦a≦1.8, 0≦b≦0.5, 0≦c≦0.05 and 0≦α≦2), LiₐNi_{1-b-c}Mn_{b}R_{c}O_{2-α}Z_{α} (0.90≦a≦1.8, 0≦b≦0.5, 0≦c≦0.05 and 0≦α≦2), LiₐNi_{1-b-c}Mn_{b}R_{c}O_{2-α}Z₂ (0.90≦a≦1.8, 0≦b≦0.5, 0≦c≦0.05 and 0≦α≦2), LiₐNi_{b}E_{c}G_{d}O₂ (0.90≦a≦1.8, 0≦b≦0.9, 0≦c≦0.5 and 0.001≦d≦0.1), LiₐNi_{b}Co_{c}Mn_{d}GeO₂ (0.90≦a≦1.8, 0≦b≦0.9, 0≦c≦0.5, 0≦d≦0.5 and 0.001≦e≦0.1), LiₐNiG_{b}O₂ (0.90≦a≦1.8 and 0.001≦b≦0.1), LiₐCoG_{b}O₂ (0.90≦a≦1.8 and 0.001≦b≦0.1), LiₐMnG_{b}O₂ (0.90≦a≦1.8 and 0.001≦b≦0.1), LiₐMn₂G_{b}O₄ (0.90≦a≦1.8 and 0.001≦b≦0.1), QO₂; QS₂; LiQS₂; V₂O₅; LiV₂O₅; LiTO₂; LiNiVO₄; Li_{(3-f)}J₂(PO₄)₃ (0≦f≦2); Li_{(3-f)}Fe₂(PO₄)₃ (0≦f≦2), and LiFePO₄. In the above formulae, A may be Ni, Co, Mn, or a combination thereof; R may be Al, Ni, Co, Mn, Cr, Fe, Mg, Sr, V, a rare earth element, or a combination thereof, D may be O, F, S, P, or a combination thereof; E is Co, Mn, or a combination thereof, Z may be F, S, P, or a combination thereof, G may be Al, Cr, Mn, Fe, Mg, La, Ce, Sr, V, or a combination thereof, Q may be Ti, Mo, Mn, or a combination thereof, T may be Cr, V, Fe, Sc, Y, or a combination thereof, and J may be V, Cr, Mn, Co, Ni, Cu, or a combination thereof.

The lithiated intercalation compound may have a coating layer on a surface, or may be mixed with a compound having a coating layer. The coating layer may include at least one coating element compound selected from the group of an oxide of a coating element, a hydroxide of a coating element, an oxyhydroxide of a coating element, an oxycarbonate of a coating element, and a hydroxyl carbonate of a coating element. The compounds for a coating layer may be amorphous or crystalline. The coating element for a coating layer may include, e.g., Mg, Al, Co, K, Na, Ca, Si, Ti, V, Sn, Ge, Ga, B, As, Zr, or a mixture thereof. The coating layer may be formed using a method that has little to no negative influence on properties of a positive active material by including these elements in the compound. For example, the method may include any suitable coating method such as spray coating, dipping, and the like.

The negative electrode 110 may include a current collector 112 and a negative active material layer 114 on the current collector 112.

The current collector 112 may include any suitable metal having high conductivity, being easily attached to the negative active material layer 114 lithium secondary battery, and having little to no reactivity within a voltage range of a lithium secondary battery. For example, the current collector 112 may include a copper foil, a nickel foil, a stainless steel foil, a titanium foil, a nickel foam, a copper foam, a polymer substrate coated with a conductive metal, or a combination thereof, but is not limited thereto.

The negative active material layer 114 may include a negative active material. For example, a carbon-based material such as crystalline carbon, amorphous carbon, or a carbon composite, that reversibly intercalates/deintercalates lithium ions, may be used. The crystalline carbon may be non-shaped, or sheet, flake, spherical, or fiber shaped natural graphite or artificial graphite. The amorphous carbon may be a soft carbon, a hard carbon, a mesophase pitch carbonization product, fired coke, and the like.

The negative active material may be an alloy of lithium metal. The alloy of lithium metal may include lithium and at least one metal selected from Na, K, Rb, Cs, Fr, Be, Mg, Ca, Sr, Si, Sb, Pb, In, Zn, Ba, Ra, Ge, Al, and Sn.

For the negative active material, a material capable of doping/dedoping lithium may also be used. The a material capable of doping/dedoping lithium may include Si, SiOₓ (0≦x≦2), a Si-Q alloy (wherein Q is an element selected from an alkali metal, an alkaline-earth metal, Group 13 to 16 elements, a transition element, a rare earth element, or a combination thereof, and not Si), Sn, SnO₂, a Sn—R alloy (wherein R is an element selected from an alkali metal, an alkaline-earth metal, Group 13 to 16 elements, a transition element, a rare earth element, or a combination thereof, and not Sn), and the like. At least one of these materials may be mixed with SiO₂. Examples of Q and R may include Mg, Ca, Sr, Ba, Ra, Sc, Y, Ti, Zr, Hf, Rf, V, Nb, Ta, Db, Cr, Mo, W, Sg, Tc, Re, Bh, Fe, Pb, Ru, Os, Hs, Rh, Ir, Pd, Pt, Cu, Ag, Au, Zn, Cd, B, Al, Ga, Sn, In, Ti, Ge, P, As, Sb, Bi, S, Se, Te, Po, or a combination thereof.

For the negative active material, a transition metal oxide such as vanadium oxide, lithium vanadium oxide, and the, like may also be used.

The positive active material layer 104 and negative active material layer 114 may further include a binder and a conductive material, in addition to the respective active materials. The binder may play a role of formation of active material into a paste, mutual adhesion of active materials, adhesion of active material to a current collector (102 or 112), or buffering of expansion and shrinkage of active materials. Examples of the binder may include polyvinylalcohol, carboxylmethylcellulose, hydroxypropylcellulose, diacetylcellulose, polyvinylchloride, carboxylated polyvinyl chloride, polyvinylfluoride, an ethylene oxide-containing polymer, polyvinylpyrrolidone, polyurethane, polytetrafluoroethylene, polyvinylidene fluoride, polyethylene, polypropylene, a styrene-butadiene rubber, an acrylated styrene-butadiene rubber, an epoxy resin, nylon, and the like, but are not limited thereto.

The binder may be included in an amount of about 0.1 wt % to about 30 wt %, based on a total weight of the active material. Within the above range, sufficient adherence between active materials and the current collector 102 and 112 may be obtained without a reduction in battery capacity.

The conductive material may be included to help improve electrode conductivity. Any suitable electrically conductive material that does not cause a chemical change may be used as the conductive material, e.g., natural graphite, artificial graphite, carbon black, acetylene black, ketjen black, a carbon fiber, a metal powder or a metal fiber including copper, nickel, aluminum, silver, and the like. At least one of a conductive material, e.g., a polyphenylene derivative, may be mixed with the forgoing conductive material.

The conductive material may be included in an amount of about 0.1 wt % to about 10 wt %, based on the total weight of the active material. Within the above range, electrochemical characteristics and energy density per weight may be adjusted to be within a desired range.

The positive electrode 100 and negative electrode 110 may be fabricated by a method including mixing an active material, a conductive material, and a binder in a solvent to prepare an active material composition or slurry, and coating the composition on the current collector 102 and 112. The solvent may include, e.g., N-methylpyrrolidone, dimethyl formamide, N,N-dimethylaminopropylamine, ethyleneoxide, tetrahydrofuran, and the like. A thickener may be further used to control viscosity of the active material slurry. For example, the thickener may include carboxyl methyl cellulose, hydroxymethyl cellulose, hydroxyethyl cellulose, hydroxypropyl cellulose, or the like.

The separator 120 may electrically separate the positive electrode 100 and negative electrode 110 and may provide a path for transport of lithium ions. The separator 120 may include a monolayer of polyethylene, polypropylene, or polyvinylidene fluoride, a multilayer formed by stacking at least two of the forgoing the monolayer, or a mixed multilayer such as a polyethylene/polypropylene mixed double layer, a polyethylene/polypropylene/polyethylene mixed triple layer, or a polypropylene/polyethylene/polypropylene mixed triple layer.

Lithium secondary batteries may be classified as lithium ion batteries, lithium ion polymer batteries, and lithium polymer batteries according to the presence of a separator and the kind of electrolyte used in the battery. The lithium secondary batteries may have a variety of shapes and sizes, and may include cylindrical, prismatic, or coin-type batteries, and may be thin film batteries or may be rather bulky in size.

In the lithium secondary battery shown in FIG. 1, lithium ions L1″ from the positive active material layer 104 during the first charge may be intercalated in the negative active material layer 114. Then, during discharge, the lithium ions L1″ may be deintercalated and intercalated in the positive active material layer 104. For example, the lithium ions L1″ may transfer energy by shuttling between the positive electrode 100 and the negative electrode 110 to perform the charge and discharge.

A passivation layer may be provided due to an electrochemical oxidation decomposition reaction of the non-aqueous electrolyte 130 at defected position on a surface of the positive electrode 100 or may be activated by performing the charge and discharge. The passivation layer may enhance impedance when lithium ions are intercalated into the positive active material layer 104. In addition, when the charge and discharge is repeated, the positive active material layer 104 may be structurally collapsed or may be chemically dissolved by the non-aqueous electrolyte 130, so that metal ions, e.g., Co, Mn, Ni, or the like, may be eluted. The reactions may deteriorate the performance of positive electrode 100 by itself In addition, the eluted metal ion may be electrodeposited on a surface of the negative electrode 110. The metal ion electrodeposited to the negative electrode 100 may have high reactivity toward the non-aqueous electrolyte 130, thereby degrading the negative electrode 100. Battery performance degradation may be further accelerated when the battery is exposed to a high temperature.

However, when the electrolyte includes the non-aqueous electrolyte additive according to an embodiment, stability of the positive electrode 100 may be enhanced by adsorbing the non-aqueous electrolyte additive in the defected position or the activated place of the surface of the positive electrode 100 to suppress the oxidation decomposition reaction of the non-aqueous electrolyte 130. In addition, the additive according to an embodiment may include a bidentate phosphorus compound, so it may form a stable cyclic chelate with the metal ion eluted from the positive electrode 100, thereby suppressing the electrodeposition of metal ion to the negative electrode 110. Accordingly, high temperature stability of the battery may also be enhanced.

The following Examples and Comparative Examples are provided in order to set forth particular details of one or more embodiments. However, it will be understood that the embodiments are not limited to the particular details described. Further, the Comparative Examples are set forth to highlight certain characteristics of certain embodiments, and are not to be construed as either limiting the scope of the invention as exemplified in the Examples or as necessarily being outside the scope of the invention in every respect.

### Preparation of Electrolyte (Experimental Examples 1 and 2 and Comparative Experimental Examples 1 and 2)

A non-aqueous electrolyte was prepared by dissolving LiPF₆ in an organic solvent having a composition of ethylene carbonate (EC):ethylmethyl carbonate (EMC):dimethyl carbonate (DMC)=2:2:6 (v:v:v) to provide a concentration of 1.3M and including an electrolyte additive according to the following Table 1. In Table 1, the amount of the electrolyte additive was based on a total weight of the resulting base electrolyte (including EC, EMC and DMC and LiPF₆).

TABLE 1

| | | | Comparative | Comparative |
|---|---|---|---|---|
| | Experimental | Experimental | Experimental | Experimental |
| | Example 1 | Example 2 | Example 1 | Example 2 |
| Additive | 6,6′-[(3,3′-di-t-butyl)- | 1,4-bis (1,3,2- | trimethylsilyl | None |
| | 5,5′-dimethoxy-1,1′- | dioxaphosphacyclopentyl)- | phosphite | |
| | biphenyl-2,2′-diyl)bis | 1,4-dioxabutane | (3 wt %) | |
| | (oxy)]bis(dibenzo[d,f] | (0.1 wt %) | | |
| | [1,3,2]dioxaphosphepin) | | | |
| | hemi ethyl acetate) | | | |
| | (BIPHEPHOS) | | | |
| | (0.1 wt %) | | | |

Fabrication of Battery Cell

A positive active material of 9.2 g of a LMO/NCM (lithium manganese oxide (LiMn₂O₄)/nickel cobalt manganese (LiNi_{0.3}Co_{0.3}Mn_{0.3}O₂)) mixture, a binder of 0.4 g of PVdF (polyvinylidene fluoride), and a conductive material of 0.4 g of denka black were added into 8 g of N-methylpyrrolidone solvent to provide a positive electrode slurry. The slurry was then coated onto an aluminum current collector. The current collector was then dried in an oven at 110° C. and compressed to provide a positive electrode.

Using a negative active material of 9.75 g of artificial graphite and a binder of 0.25 g of styrene butadiene rubber/carboxymethyl cellulose, a negative electrode slurry was prepared and coated on a copper current collector. The current collector was then dried in an oven at 110° C. and compressed to provide a negative electrode.

A polyethylene/polypropylene film separator was interposed between the obtained positive electrode and negative electrode to provide a pouch cell, and then the pouch cell was injected with each non-aqueous electrolyte obtained from Table 1 to provide a pouch lithium secondary battery cell.

Performance Evaluation

Measurement of capacity and power retention after 90 cycles at 25° C.

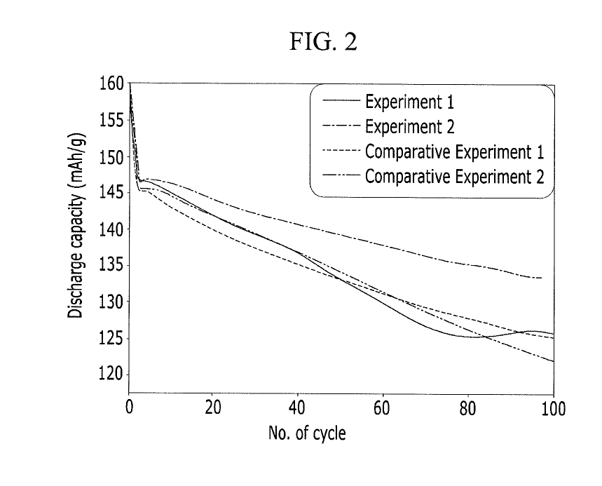

The obtained pouch cells were charged and discharged at 1 C at 25° C. FIG. 2 illustrates a graph showing results of internal resistance and capacitance retention of Experimental Examples 1 and 2 and Comparative Experimental Examples 1 and 2 after 90 cycles. Referring to FIG. 2, Experimental Example 2 exhibited remarkably improved cycle characteristics, compared to Comparative Experimental Examples 1 and 2. Experimental Example 1 exhibited similar or slightly improved cycle characteristics, compared to Comparative Experimental Examples 1 and 2.

By way of summation and review, a lithium secondary battery may include a positive electrode, a negative electrode, and an electrolyte. Upon repeated charge and discharge of battery, a structure of a positive active material may be collapsed, thereby deteriorating battery performance. In addition, a metal ion eluted from a surface of the positive electrode during the structure collapse of positive electrode may be electrodeposited to the negative electrode to deteriorate the negative electrode. The battery performance deterioration may be accelerated when increasing a potential of the positive electrode or exposing the battery to a high temperature.

The embodiments provide a non-aqueous electrolyte additive for enhancing stability and high-temperature stability of a positive electrode for a lithium secondary battery.

The embodiments also provide a non-aqueous electrolyte for enhancing stability and high-temperature stability of a positive electrode of a lithium secondary battery.

The embodiments also provide a lithium secondary battery having enhanced stability and high temperature stability of a positive electrode.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. A non-aqueous electrolyte additive for a lithium secondary battery, the additive comprising a bidentate phosphorus compound represented by the following Chemical Formula 1, 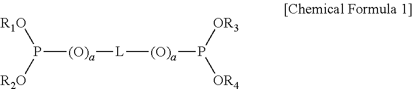
wherein:
a is independently 0 or 1, and
R₁, R₂, R₃, and R₄ are each independently selected from the group of a substituted or unsubstituted C1 to C15 alkyl group, a substituted or unsubstituted C3 to C15 alkylsilyl group, a substituted or unsubstituted C3 to C15 cycloalkyl group, a substituted or unsubstituted C3 to C15 heterocycloalkyl group, a substituted or unsubstituted C6 to C15 aryl group, a substituted or unsubstituted C2 to C15 heteroaryl group, and a substituted or unsubstituted C2 to C15 alkenyl group, or
R₁, R₂, R₃ and R₄ are independently linked together to provide one selected from the group of an oxygen-containing substituted or unsubstituted C2 to C15 heterocycloalkyl group and an oxygen-containing substituted or unsubstituted C2 to C15 heteroaryl group, and
L is selected from the group of a C2 to C15 alkenylene group, a covalent bond bridging group represented by the following Chemical Formula 2, and a covalent bond bridging group represented by the following Chemical Formula 3, 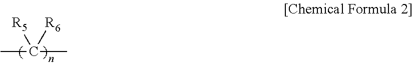
wherein:
n is an integer of 1 to about 4, and
R₅ and R₆ are linked together to provide one selected from the group of a substituted or unsubstituted C3 to C15 cycloalkyl group, a substituted or unsubstituted C3 to C15 heterocycloalkyl group, a substituted or unsubstituted C3 to C20 aryl group, and a substituted or unsubstituted C2 to C20 heteroaryl group, or
R₅ and R₆ are each independently selected from the group of hydrogen, an amine group, a cyano group, a thiolate group, a substituted or unsubstituted C1 to C15 alkyl group, —OR₇, —SiR₈R₉R₁₀, —OSiR₁₁R₁₂R₁₃, —SR₁₄, —SOR₁₅, —BR₁₆R₁₇, —OBR₁₈R₁₉, —OCOR₂₀ and —COR₂₁, in which R₇ to R₁₉ are each independently a substituted or unsubstituted C1 to C15 alkyl group, a substituted or unsubstituted C3 to C15 cycloalkyl group, a substituted or unsubstituted C3 to C15 heterocycloalkyl group, a substituted or unsubstituted C6 to C15 aryl group, or a substituted or unsubstituted C2 to C15 heteroaryl group,
(CH₂)_{b}-(A,X)Ar(Y,D)-(CH₂)_{b} [Chemical Formula 3]
wherein,
b is independently 0 to about 6,
Ar is a substituted or unsubstituted C6 to C30 aryl group or a substituted or unsubstituted C1 to C30 heteroaryl group, and
A, X, Y, and D are a substituent of Ar, and are each independently selected from the group of hydrogen, a substituted or unsubstituted C1 to C15 alkyl group, a substituted or unsubstituted C3 to C15 cycloalkyl group, a substituted or unsubstituted C3 to C15 heterocycloalkyl group, a substituted or unsubstituted C6 to C20 aryl group, a substituted or unsubstituted C2 to C20 heteroaryl group, a substituted or unsubstituted C2 to C15 alkenyl group, a substituted or unsubstituted C2 to C15 alkynyl group, a halo group, a cyano group, a nitro group, —OH, —OR₂₀, —COR₂₁, —COOR₂₂, —SH, —SR₂₃, —COSR₂₄, —NH₂, —NHR₂₅, —NR₂₆R₂₇, —NO₂, —CN, —CONH₂, —CONHR₂₈, and —CONR₂₉R₃₀, in which R₂₀ to R₃₀ are each independently a C1 to C15 alkyl group.

2. The non-aqueous electrolyte additive as claimed in claim 1, wherein the compound represented by Chemical Formula 1 forms a cyclic chelate with a metal ion eluted from a positive electrode for a lithium secondary battery including the additive.

3. The non-aqueous electrolyte additive as claimed in claim 1, wherein the compound represented by Chemical Formula 1 includes at least one of 1,3-bis(diphenoxyphosphine)propane, 1,3-bis(di(trimethylsiloxy)phosphine)methane, bis[1,3,2-dioxaphospholanyl-2]methane, 1,3-bis-[1,3,2-dioxaphospholanyl-2]propane, 1,2-bis[di(t-butoxy)phosphinomethyl]benzene, 1,2-bis[di(trimethylsiloxy)phosphinomethyl]benzene, 1,2-bis[di(t-butoxy)phosphinomethyl]-3,6-diphenyl-4,5-dimethylbenzene, 1,2-bis[di(t-butoxy)phosphinomethyl]-4,5-diphenylbenzene, 1,2-bis(decyloxy phosphorous acid)ethane, 1,4-bis(di(trimethylsiloxy)phosphino)-1,4-dioxabutane, phosphorous acid 2-(diphenoxy-phosphanyloxy)-ethyl ester diphenyl ester, phosphorous acid diallyl ester 2-(bis-allyloxy-phosphanyloxy)-ethyl ester, 1,4-bis(1,3,2-dioxaphosphacyclopentyl)-1,4-dioxabutane), 2-[2-(1,3-dioxa-6-aza-2-phospha-phenalen-2-yloxy)-ethoxy]-1,3-dioxa-6-aza-2-phospha-phenalene), phosphorous acid 2-amino-2-(diethoxy-phosphanyloxy)-ethyl ester diethyl ester, phosphorous acid 2-cyano-2-(diethoxy-phosphanyloxy)-ethyl ester diethyl ester, phosphorous acid 2-(diethoxy-phosphanyloxy)-2-mercapto-ethyl ester diethyl ester, phosphorous acid 2-(diethoxy-phosphanyloxy)-butyl ester diethyl ester, phosphorous acid 2-(diethoxy-phosphanyloxy)-1-methyl-propyl ester diethyl ester, phosphorous acid 2-(diethoxy-phosphanyloxy)-2-ethoxy-ethyl ester diethyl ester, phosphorous acid 2-(tert-butyl-dimethyl-silanyl)-2-(diethoxy-phosphanyloxy)-ethyl ester diethyl ester, phosphorous acid 2-(diethoxy-phosphanyloxy)-2-trimethylsilanyloxy-ethyl ester diethyl ester, phosphorous acid 2-(diethoxy-phosphanyloxy)-2-ethylsulfanyl-ethyl ester diethyl ester, phosphorous acid 2-(diethoxy-phosphanyloxy)-2-(4,4,5,5-tetramethyl-[1,3,2]dioxaborolan-2-yl)-ethyl ester diethyl ester, carbonic acid 1,2-bis-(diethoxy-phosphanyloxy)-ethyl ester methyl ester, acetic acid 1,2-bis-(diethoxy-phosphanyloxy)-ethyl ester, and compounds represented by the following Chemical Formulas 11 to 16, in which iPr denotes isopropyl and Me denotes methyl,

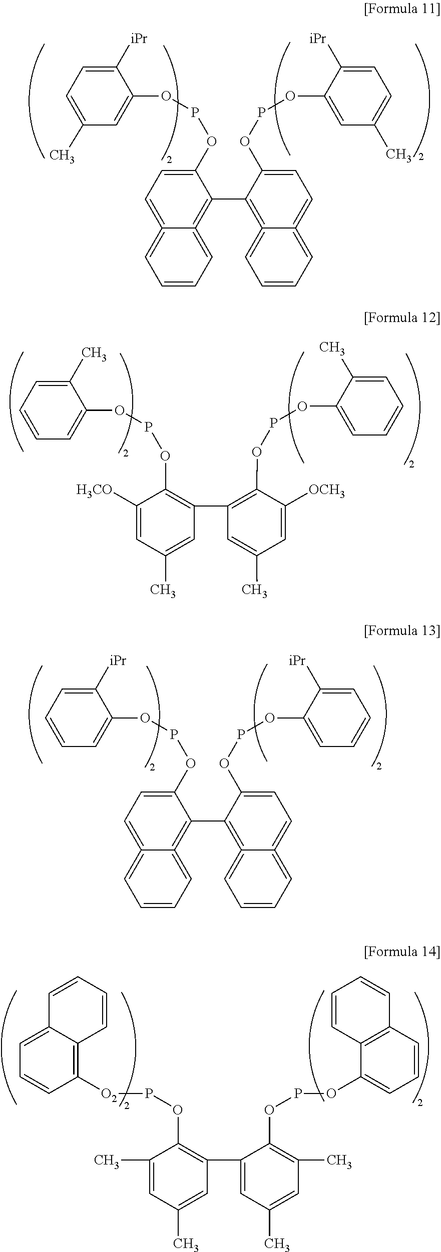

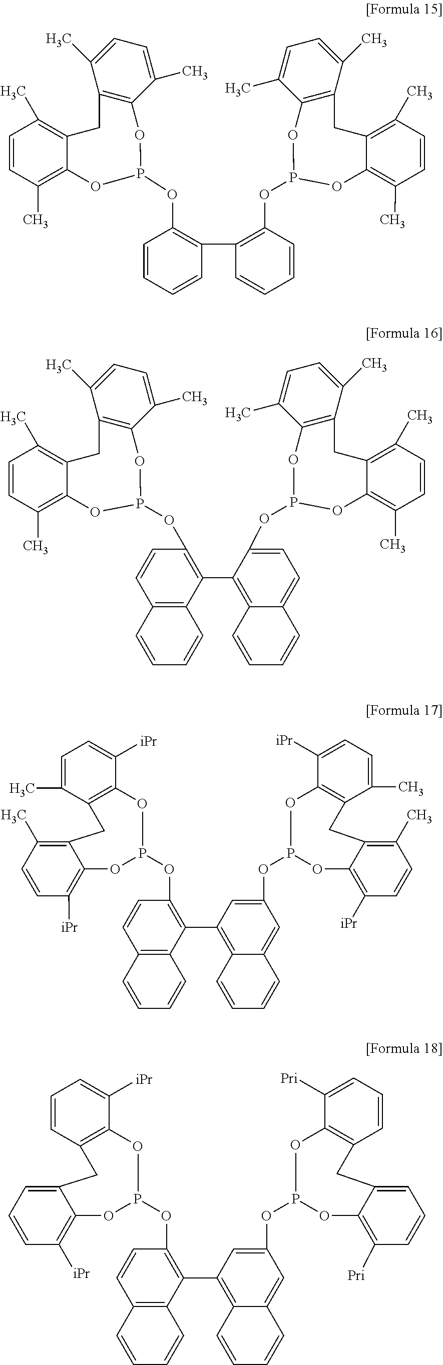

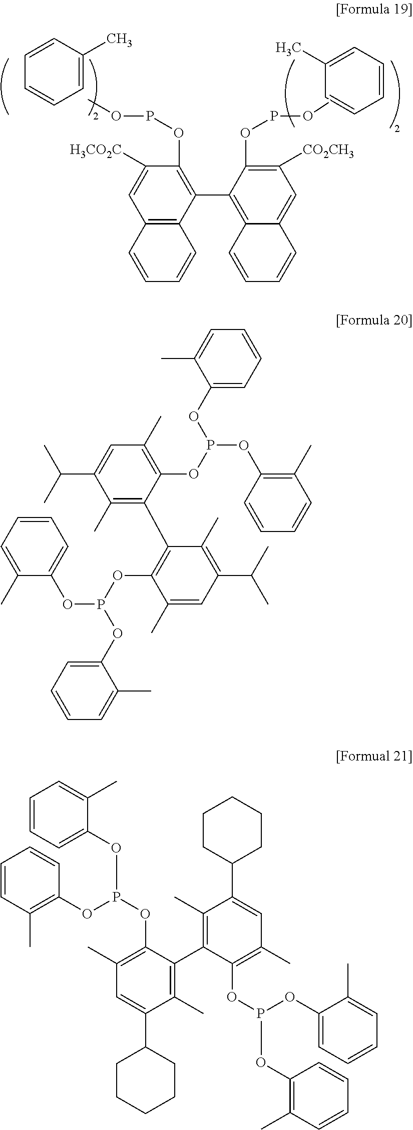

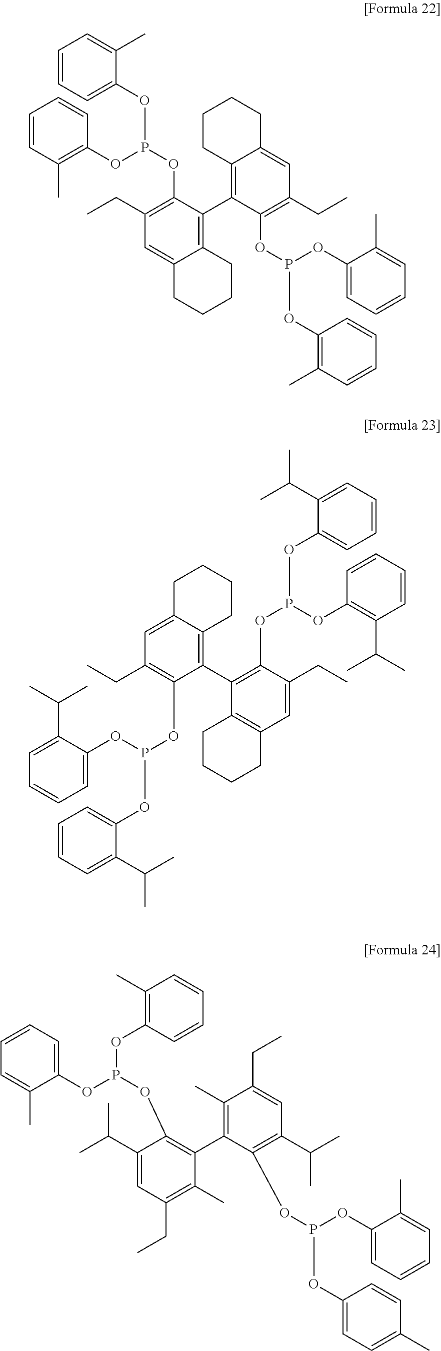 [SMILES: CCc1cc2c(c(-c3c4c(cc(CC)c3OP(Oc3ccccc3C)Oc3ccccc3C)CCCC4)c1OP(Oc1ccccc1C)Oc1ccccc1C)CCCC2 ; CCc1cc2c(c(-c3c4c(cc(CC)c3OP(Oc3ccccc3C(C)C)Oc3ccccc3C(C)C)CCCC4)c1OP(Oc1ccccc1C(C)C)Oc1ccccc1C(C)C)CCCC2 ; CCc1cc(C(C)C)c(OP(Oc2ccc(C)cc2)Oc2ccccc2C)c(-c2c(C)c(CC)cc(C(C)C)c2OP(Oc2ccccc2C)Oc2ccccc2C)c1C]

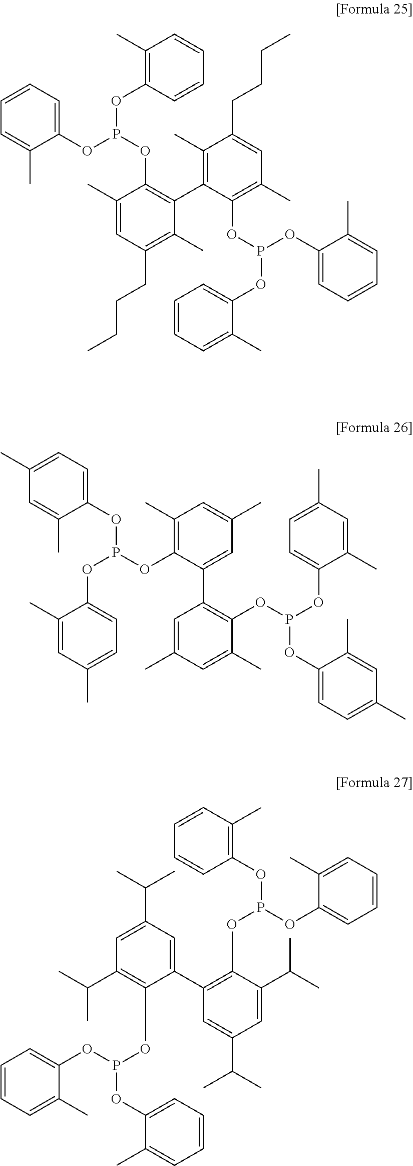

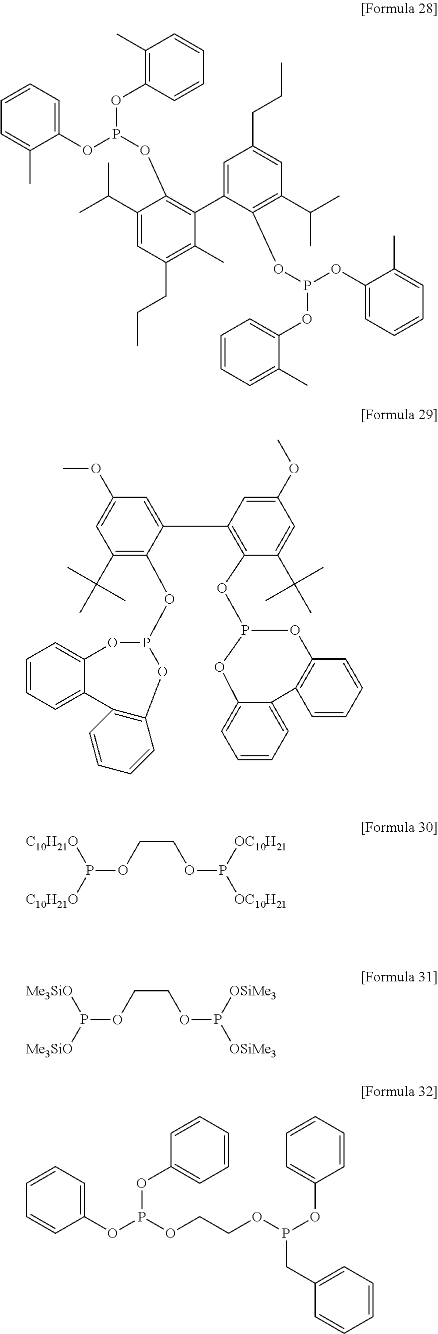

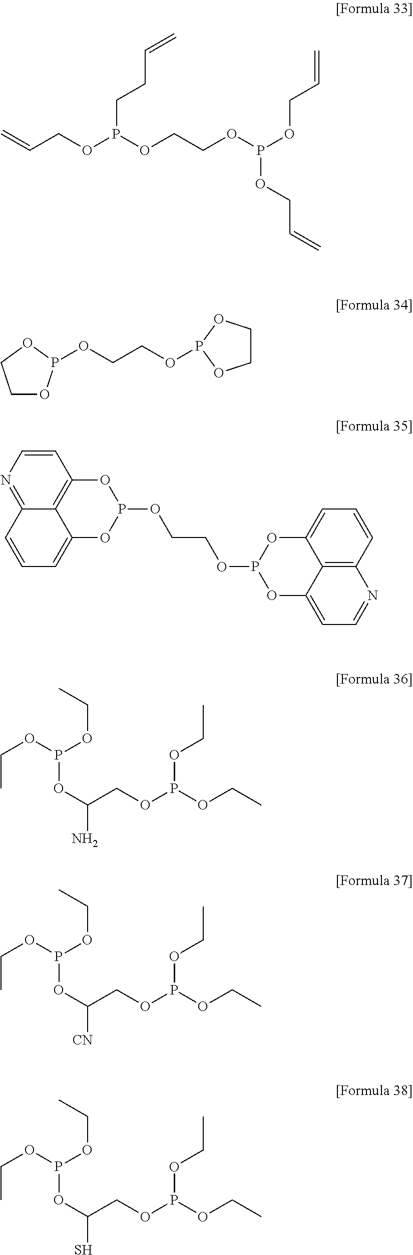

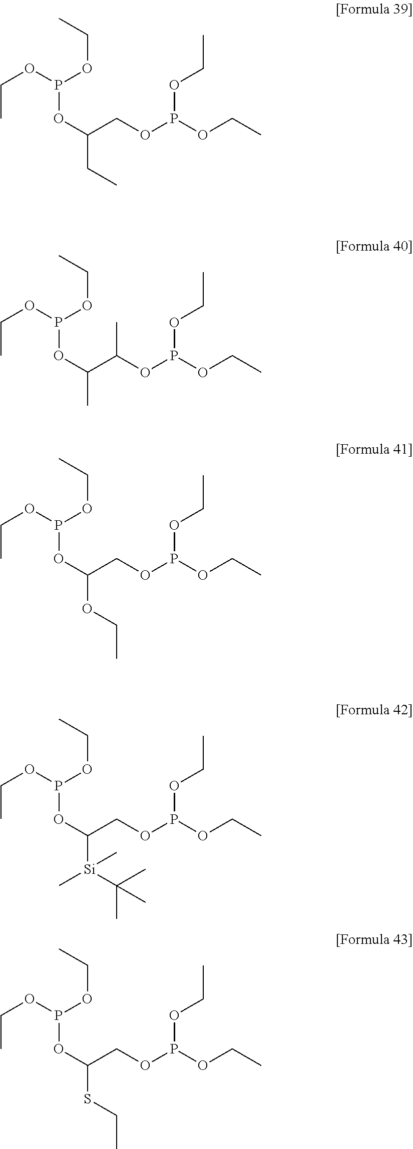 [SMILES: CCOP(OCC)OCC(CC)OP(OCC)OCC ; CCOP(OCC)OC(C)C(C)OP(OCC)OCC ; CCOC(COP(OCC)OCC)OP(OCC)OCC ; CCOP(OCC)OCC(OP(OCC)OCC)[Si](C)(C)C(C)(C)C ; CCOP(OCC)OCC(OP(OCC)OCC)SCC]

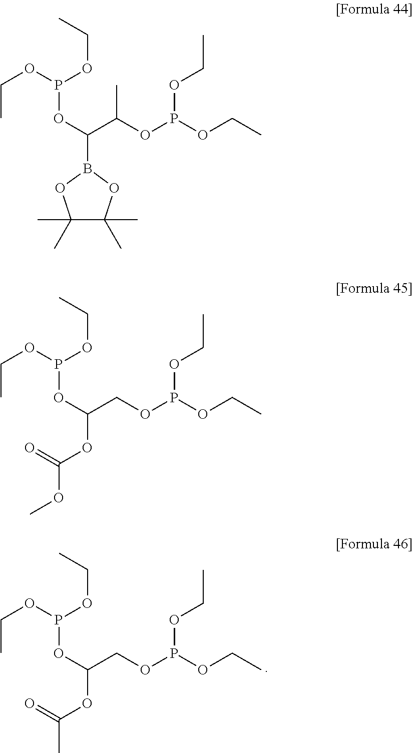 [SMILES: CCOP(OCC)OC(C)C(OP(OCC)OCC)B1OC(C)(C)C(C)(C)O1 ; CCOP(OCC)OCC(OC(=O)OC)OP(OCC)OCC ; CCOP(OCC)OCC(OC(C)=O)OP(OCC)OCC]

4. The non-aqueous electrolyte additive as claimed in claim 1, wherein the compound represented by Chemical Formula 1 includes at least one of 6,6′-[(3,3′-di-t-butyl)-5,5′-dimethoxy-1,1′-biphenyl-2,2′-diyl)bis(oxy)]bis(dibenzo[d,f][1,3,2]dioxaphosphepin)hemi ethyl acetate) and 1,4-bis(1,3,2-dioxaphosphacyclopentyl)-1,4-di oxabutane.

5. A non-aqueous electrolyte for a lithium secondary battery, the electrolyte comprising:
a base electrolyte including a non-aqueous organic solvent and a lithium salt dissolved in the non-aqueous organic solvent; and
a bidentate phosphorus compound represented by the following Chemical Formula 1, 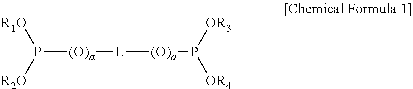
wherein:
a is independently 0 or 1, and
R₁, R₂, R₃, and R₄ are each independently selected from the group of a substituted or unsubstituted C1 to C15 alkyl group, a substituted or unsubstituted C3 to C15 alkylsilyl group, a substituted or unsubstituted C3 to C15 cycloalkyl group, a substituted or unsubstituted C3 to C15 heterocycloalkyl group, a substituted or unsubstituted C6 to C15 aryl group, a substituted or unsubstituted C2 to C15 heteroaryl group, and a substituted or unsubstituted C2 to C15 alkenyl group, or
R₁, R₂, R₃ and R₄ are independently linked together to provide one selected from the group of an oxygen-containing substituted or unsubstituted C2 to C15 heterocycloalkyl group and an oxygen-containing substituted or unsubstituted C2 to C15 heteroaryl group, and
L is selected from the group of a C2 to C15 alkenylene group, a covalent bond bridging group represented by the following Chemical Formula 2, and a covalent bond bridging group represented by the following Chemical Formula 3, 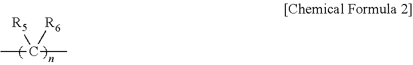
wherein:
n is an integer of 1 to about 4, and
R₅ and R₆ are linked together to provide one selected from the group of a substituted or unsubstituted C3 to C15 cycloalkyl group, a substituted or unsubstituted C3 to C15 heterocycloalkyl group, a substituted or unsubstituted C3 to C20 aryl group, and a substituted or unsubstituted C2 to C20 heteroaryl group, or
R₅ and R₆ are each independently selected from the group of hydrogen, an amine group, a cyano group, a thiolate group, a substituted or unsubstituted C1 to C15 alkyl group, —OR₇, —SiR₈R₉R₁₀, —OSiR₁₁R₁₂R₁₃, —SR₁₄, —SOR₁₅, —BR₁₆R₁₇, —OBR₁₈R₁₉, —OCOR₂₀ and —COR₂₁, in which R₇ to R₁₉ are each independently a substituted or unsubstituted C1 to C15 alkyl group, a substituted or unsubstituted C3 to C15 cycloalkyl group, a substituted or unsubstituted C3 to C15 heterocycloalkyl group, a substituted or unsubstituted C6 to C15 aryl group, or a substituted or unsubstituted C2 to C15 heteroaryl group,
(CH₂)_{b}-(A,X)Ar(Y,D)-(CH₂)_{b} [Chemical Formula 3]
wherein,
b is independently 0 to about 6,
Ar is a substituted or unsubstituted C6 to C30 aryl group or a substituted or unsubstituted C1 to C30 heteroaryl group, and
A, X, Y, and D are a substituent of Ar, and are each independently selected from the group of hydrogen, a substituted or unsubstituted C1 to C15 alkyl group, a substituted or unsubstituted C3 to C15 cycloalkyl group, a substituted or unsubstituted C3 to C15 heterocycloalkyl group, a substituted or unsubstituted C6 to C20 aryl group, a substituted or unsubstituted C2 to C20 heteroaryl group, a substituted or unsubstituted C2 to C15 alkenyl group, a substituted or unsubstituted C2 to C15 alkynyl group, a halo group, a cyano group, a nitro group, —OH, —OR₂₀, —COR₂₁, —COOR₂₂, —SH, —SR₂₃, —COSR₂₄, —NH₂, —NHR₂₅, —NR₂₆R₂₇, —NO₂, —CN, —CONH₂, —CONHR₂₈, and —CONR₂₉R₃₀, in which R₂₀ to R₃₀ are each independently a C1 to C15 alkyl group.

6. The non-aqueous electrolyte as claimed in claim 5, wherein the compound represented by Chemical Formula 1 forms a cyclic chelate with a metal ion eluted from a positive electrode of lithium secondary battery including the non-aqueous electrolyte additive.

7. The non-aqueous electrolyte as claimed in claim 5, wherein the compound represented by Chemical Formula 1 is included in the electrolyte in an amount of about 0.01 wt % to about 20 wt %, based on a total weight of the base electrolyte.

8. The non-aqueous electrolyte additive as claimed in claim 5, wherein the compound represented by Chemical Formula 1 includes at least one of 1,3-bis(diphenoxyphosphine)propane, 1,3-bis(di(trimethylsiloxy)phosphine)methane, bis[1,3,2-dioxaphospholanyl-2]methane, 1,3-bis-[1,3,2-dioxaphospholanyl-2]propane, 1,2-bis[di(t-butoxy)phosphinomethyl]benzene, 1,2-bis[di(trimethylsiloxy)phosphinomethyl]benzene, 1,2-bis[di(t-butoxy)phosphinomethyl]-3,6-diphenyl-4,5-dimethylbenzene, 1,2-bis[di(t-butoxy)phosphinomethyl]-4,5-diphenylbenzene, 1,2-bis(decyloxy phosphorous acid)ethane, 1,4-bis(di(trimethylsiloxy)phosphino)-1,4-dioxabutane, phosphorous acid 2-(diphenoxy-phosphanyloxy)-ethyl ester diphenyl ester, phosphorous acid diallyl ester 2-(bis-allyloxy-phosphanyloxy)-ethyl ester, 1,4-bis(1,3,2-dioxaphosphacyclopentyl)-1,4-dioxabutane), 2-[2-(1,3-dioxa-6-aza-2-phospha-phenalen-2-yloxy)-ethoxy]-1,3-dioxa-6-aza-2-phospha-phenalene), phosphorous acid 2-amino-2-(diethoxy-phosphanyloxy)-ethyl ester diethyl ester, phosphorous acid 2-cyano-2-(diethoxy-phosphanyloxy)-ethyl ester diethyl ester, phosphorous acid 2-(diethoxy-phosphanyloxy)-2-mercapto-ethyl ester diethyl ester, phosphorous acid 2-(diethoxy-phosphanyloxy)-butyl ester diethyl ester, phosphorous acid 2-(diethoxy-phosphanyloxy)-1-methyl-propyl ester diethyl ester, phosphorous acid 2-(diethoxy-phosphanyloxy)-2-ethoxy-ethyl ester diethyl ester, phosphorous acid 2-(tert-butyl-dimethyl-silanyl)-2-(diethoxy-phosphanyloxy)-ethyl ester diethyl ester, phosphorous acid 2-(diethoxy-phosphanyloxy)-2-trimethylsilanyloxy-ethyl ester diethyl ester, phosphorous acid 2-(diethoxy-phosphanyloxy)-2-ethylsulfanyl-ethyl ester diethyl ester, phosphorous acid 2-(diethoxy-phosphanyloxy)-2-(4,4,5,5-tetramethyl-[1,3,2]dioxaborolan-2-yl)-ethyl ester diethyl ester, carbonic acid 1,2-bis-(diethoxy-phosphanyloxy)-ethyl ester methyl ester, acetic acid 1,2-bis-(diethoxy-phosphanyloxy)-ethyl ester, and compounds represented by the following Chemical Formulas 11 to 46, in which iPr denotes isopropyl and Me denotes methyl,

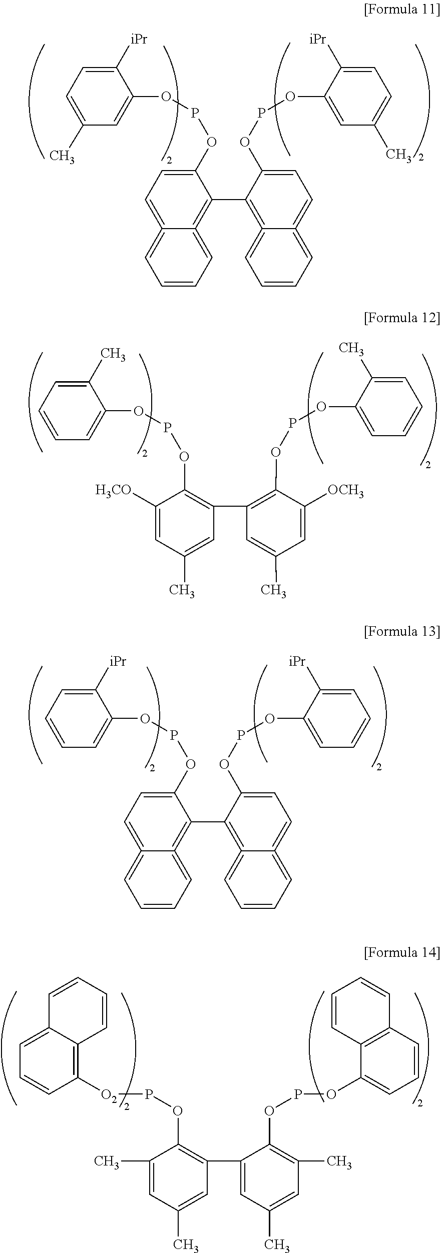

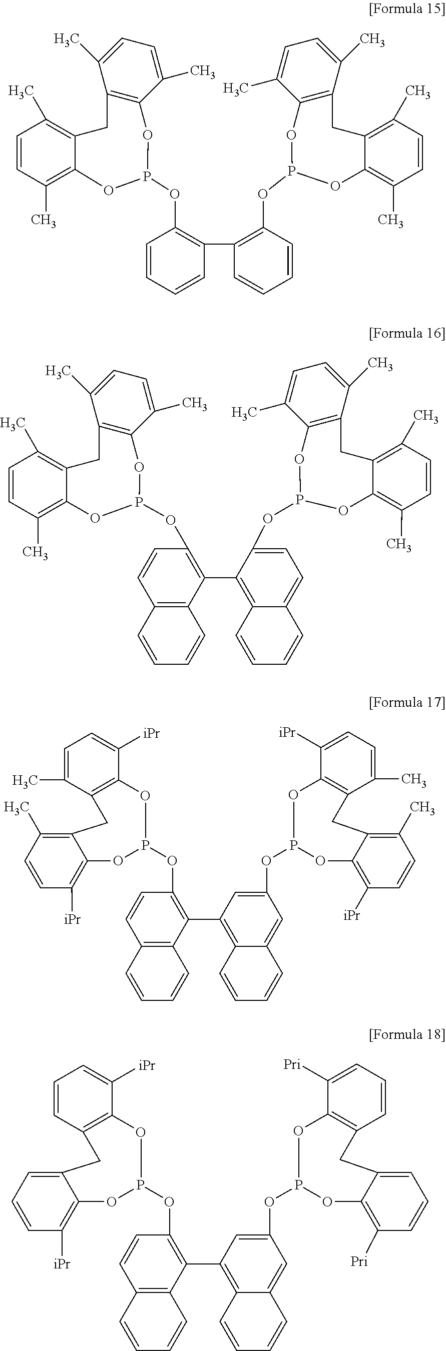

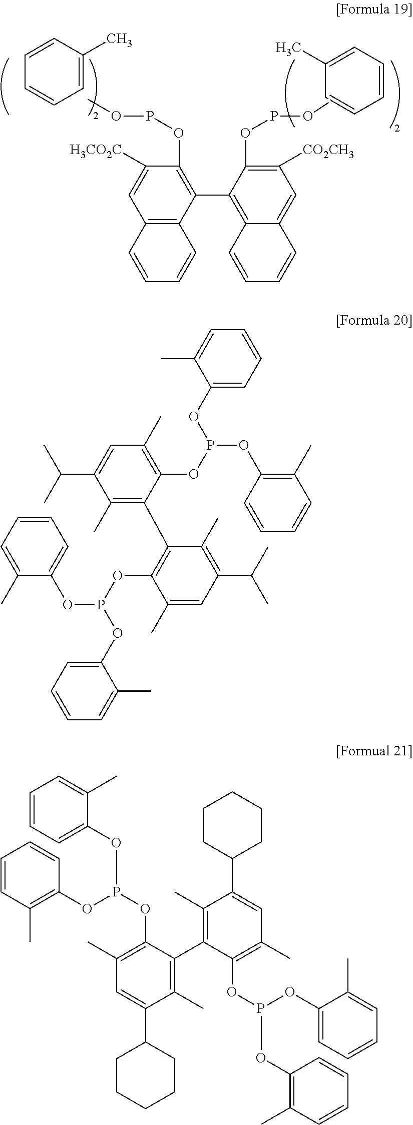

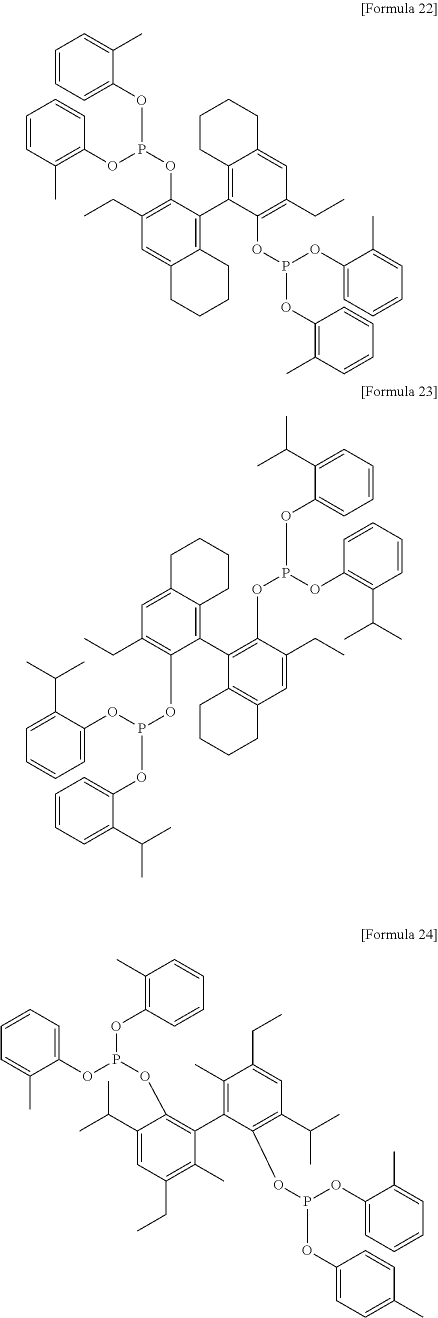

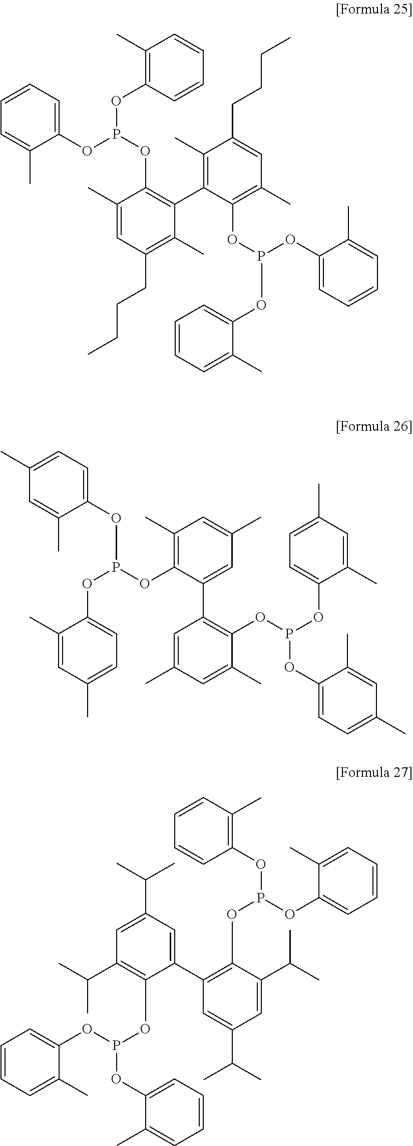

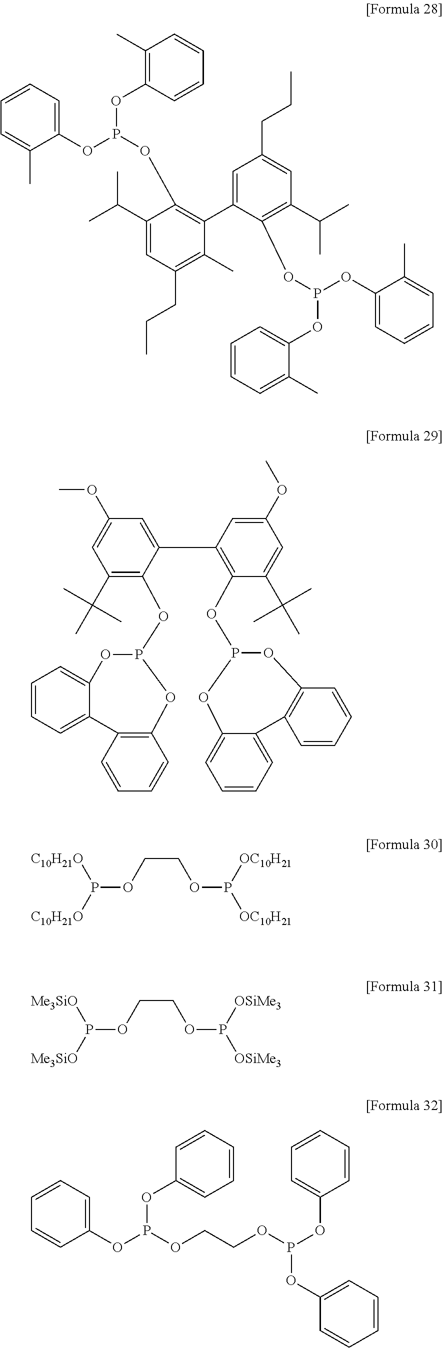

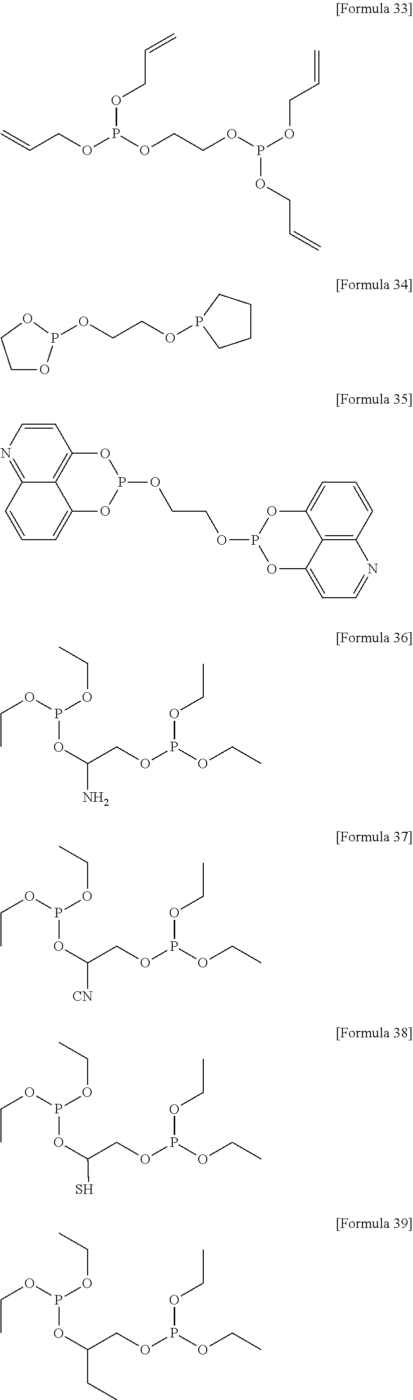

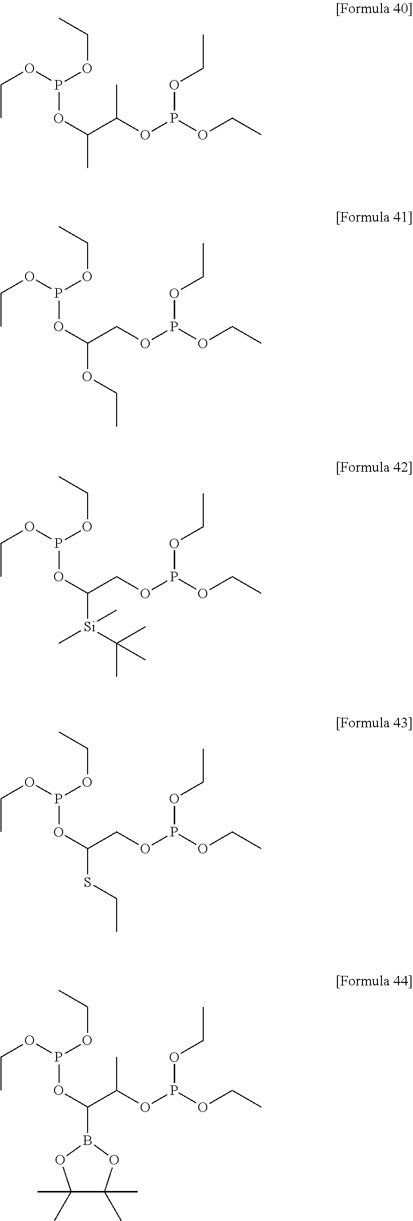 [SMILES: CCOP(OCC)OC(C)C(C)OP(OCC)OCC ; CCOC(COP(OCC)OCC)OP(OCC)OCC ; CCOP(OCC)OCC(OP(OCC)OCC)[Si](C)(C)C(C)(C)C ; CCOP(OCC)OCC(OP(OCC)OCC)SCC ; CCOP(OCC)OC(C)C(OP(OCC)OCC)B1OC(C)(C)C(C)(C)O1]

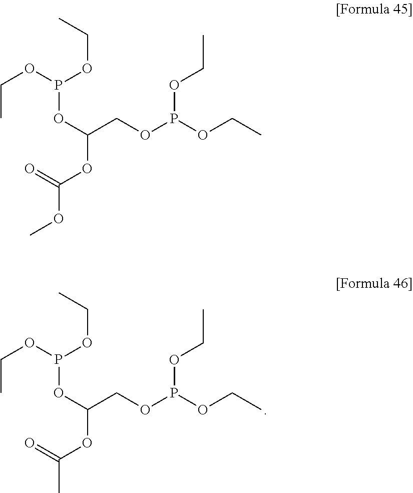 [SMILES: CCOP(OCC)OCC(OC(=O)OC)OP(OCC)OCC ; CCOP(OCC)OCC(OC(C)=O)OP(OCC)OCC]

9. The non-aqueous electrolyte additive as claimed in claim 5, wherein the compound represented by Chemical Formula 1 includes at least one of 6,6′-[(3,3′-di-t-butyl)-5,5′-dimethoxy-1,1′-biphenyl-2,2′-diyl)bis(oxy)]bis(dibenzo[d,f][1,3,2]dioxaphosphepin)hemi ethyl acetate) and 1,4-bis(1,3,2-dioxaphosphacyclopentyl)-1,4-dioxabutane.

10. A lithium secondary battery, comprising:
a positive electrode including a positive active material layer capable of intercalating and deintercalating lithium;
a negative electrode including a negative active material layer;
a separator between the positive electrode and negative electrode; and
the non-aqueous electrolyte as claimed in claim 5.

11. The lithium secondary battery as claimed in claim 10, wherein the positive active material includes one selected from LiₐA_{1-b}R_{b}D₂ (0.90≦a≦1.8 and 0≦b≦0.5), LiₐE_{1-b}R_{b}O_{2-c}D_{c} (0.90≦a≦1.8, 0≦b≦0.5 and 0≦c≦0.05), LiE_{2-b}R_{b}O_{4-c}D_{c} (0≦b≦0.5, 0≦c≦0.05), LiₐNi_{1-b-c}Co_{b}R_{c}D_{α} (0.90≦a≦1.8, 0≦b≦0.5, 0≦c≦0.05 and 0≦α≦2), LiₐNi_{1-b-c}Co_{b}R_{c}O_{2-α}Z_{α} (0.90≦a≦1.8, 0≦b≦0.5, 0≦c≦0.05 and 0≦α≦2), LiₐNi_{1-b-c}Co_{b}R_{c}O_{2-α}Z₂ (0.90≦a≦1.8, 0≦b≦0.5, 0≦c≦0.05 and 0≦α≦2), LiₐNi_{1-b-c}Mn_{b}R_{c}D_{α} (0.90≦a≦1.8, 0≦b≦0.5, 0≦c≦0.05 and 0≦α≦2), LiₐNi_{1-b-c}Mn_{b}R_{c}O_{2-α}Z_{α} (0.90≦a≦1.8, 0≦b≦0.5, 0≦c≦0.05 and 0≦α≦2), LiₐNi_{1-b-c}Mn_{b}R_{c}O_{2-α}Z₂ (0.90≦a≦1.8, 0≦b≦0.5, 0≦c≦0.05 and 0≦α≦2), LiₐNi_{b}E_{c}G_{d}O₂ (0.90≦a≦1.8, 0≦b≦0.9, 0≦c≦0.5 and 0.001≦d≦0.1), LiₐNi_{b}Co_{c}Mn_{d}GeO₂ (0.90≦a≦1.8, 0≦b≦0.9, 0≦c≦0.5, 0≦d≦0.5 and 0.001≦e≦0.1), LiₐNiG_{b}O₂ (0.90≦a≦1.8 and 0.001≦b≦0.1), LiₐCoG_{b}O₂ (0.90≦a≦1.8 and 0.001≦b≦0.1), LiₐMnG_{b}O₂ (0.90≦a≦1.8 and 0.001≦b≦0.1), LiₐMn₂G_{b}O₄ (0.90≦a≦1.8 and 0.001≦b≦0.1), QO₂; QS₂; LiQS₂; V₂O₅; LiV₂O₅; LiTO₂; LiNiVO₄; Li_{(3-f)}J₂(PO₄)₃ (0≦f≦2); Li_{(3-f)}Fe₂(PO₄)₃ (0≦f≦2), LiFePO₄, and a combination thereof,
wherein, in the above formulae, A is Ni, Co, Mn, or a combination thereof; R is Al, Ni, Co, Mn, Cr, Fe, Mg, Sr, V, a rare earth element, or a combination thereof, D is O, F, S, P, or a combination thereof; E is Co, Mn, or a combination thereof, Z is F, S, P, or a combination thereof, G is Al, Cr, Mn, Fe, Mg, La, Ce, Sr, V, or a combination thereof, Q is Ti, Mo, Mn, or a combination thereof, T is Cr, V, Fe, Sc, Y, or a combination thereof, and J is V, Cr, Mn, Co, Ni, Cu, or a combination thereof.

12. A lithium secondary battery, comprising:
a positive electrode including a positive active material layer capable of intercalating and deintercalating lithium;
a negative electrode including a negative active material layer;
a separator between the positive electrode and negative electrode; and
an electrolyte including a bidentate phosphorus compound represented by the following Chemical Formula 1, the compound represented by Chemical Formula 1 linking to a surface of the positive electrode or forming a chelate with a metal ion eluted from the positive active material, 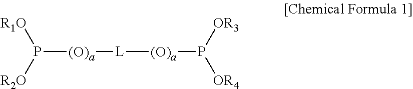
wherein:
a is independently 0 or 1, and
R₁, R₂, R₃, and R₄ are each independently selected from the group of a substituted or unsubstituted C1 to C15 alkyl group, a substituted or unsubstituted C3 to C15 alkylsilyl group, a substituted or unsubstituted C3 to C15 cycloalkyl group, a substituted or unsubstituted C3 to C15 heterocycloalkyl group, a substituted or unsubstituted C6 to C15 aryl group, a substituted or unsubstituted C2 to C15 heteroaryl group, and a substituted or unsubstituted C2 to C15 alkenyl group, or
R₁, R₂, R₃ and R₄ are independently linked together to provide one selected from the group of an oxygen-containing substituted or unsubstituted C2 to C15 heterocycloalkyl group and an oxygen-containing substituted or unsubstituted C2 to C15 heteroaryl group, and
L is selected from the group of a C2 to C15 alkenylene group, a covalent bond bridging group represented by the following Chemical Formula 2, and a covalent bond bridging group represented by the following Chemical Formula 3, 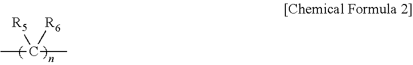
wherein:
n is an integer of 1 to about 4, and
R₅ and R₆ are linked together to provide one selected from the group of a substituted or unsubstituted C3 to C15 cycloalkyl group, a substituted or unsubstituted C3 to C15 heterocycloalkyl group, a substituted or unsubstituted C3 to C20 aryl group, and a substituted or unsubstituted C2 to C20 heteroaryl group, or
R₅ and R₆ are each independently selected from the group of hydrogen, an amine group, a cyano group, a thiolate group, a substituted or unsubstituted C1 to C15 alkyl group, —OR₇, —SiR₈R₉R₁₀, —OSiR₁₁R₁₂R₁₃, —SR₁₄, —SOR₁₅, —BR₁₆R₁₇, —OBR₁₈R₁₉, —OCOR₂₀ and —COR₂₁, in which R₇ to R₁₉ are each independently a substituted or unsubstituted C1 to C15 alkyl group, a substituted or unsubstituted C3 to C15 cycloalkyl group, a substituted or unsubstituted C3 to C15 heterocycloalkyl group, a substituted or unsubstituted C6 to C15 aryl group, or a substituted or unsubstituted C2 to C15 heteroaryl group,
(CH₂)_{b}-(A,X)Ar(Y,D)-(CH₂)_{b} [Chemical Formula 3]
wherein,
b is independently 0 to about 6,
Ar is a substituted or unsubstituted C6 to C30 aryl group or a substituted or unsubstituted C1 to C30 heteroaryl group, and
A, X, Y, and D are a substituent of Ar, and are each independently selected from the group of hydrogen, a substituted or unsubstituted C1 to C15 alkyl group, a substituted or unsubstituted C3 to C15 cycloalkyl group, a substituted or unsubstituted C3 to C15 heterocycloalkyl group, a substituted or unsubstituted C6 to C20 aryl group, a substituted or unsubstituted C2 to C20 heteroaryl group, a substituted or unsubstituted C2 to C15 alkenyl group, a substituted or unsubstituted C2 to C15 alkynyl group, a halo group, a cyano group, a nitro group, —OH, —OR₂₀, —COR₂₁, —COOR₂₂, —SH, —SR₂₃, —COSR₂₄, —NH₂, —NHR₂₅, —NR₂₆R₂₇, —NO₂, —CN, —CONH₂, —CONHR₂₈, and —CONR₂₉R₃₀, in which R₂₀ to R₃₀ are each independently a C1 to C15 alkyl group.

13. The lithium secondary battery as claimed in claim 12, wherein the chelate is a cyclic chelate.

14. The lithium secondary battery as claimed in claim 12, wherein the compound represented by Chemical Formula 1 is included in the electrolyte in an amount of about 0.01 wt % to about 20 wt %, based on a total weight of the electrolyte.

15. The non-aqueous electrolyte additive as claimed in claim 12, wherein the compound represented by Chemical Formula 1 includes at least one of 1,3-bis(diphenoxyphosphine)propane, 1,3-bis(di(trimethylsiloxy)phosphine)methane, bis[1,3,2-dioxaphospholanyl-2]methane, 1,3-bis-[1,3,2-dioxaphospholanyl-2]propane, 1,2-bis[di(t-butoxy)phosphinomethyl]benzene, 1,2-bis[di(trimethylsiloxy)phosphinomethyl]benzene, 1,2-bis[di(t-butoxy)phosphinomethyl]-3,6-diphenyl-4,5-dimethylbenzene, 1,2-bis[di(t-butoxy)phosphinomethyl]-4,5-diphenylbenzene, 1,2-bis(decyloxy phosphorous acid)ethane, 1,4-bis(di(trimethylsiloxy)phosphino)-1,4-dioxabutane, phosphorous acid 2-(diphenoxy-phosphanyloxy)-ethyl ester diphenyl ester, phosphorous acid diallyl ester 2-(bis-allyloxy-phosphanyloxy)-ethyl ester, 1,4-bis(1,3,2-dioxaphosphacyclopentyl)-1,4-dioxabutane), 2-[2-(1,3-dioxa-6-aza-2-phospha-phenalen-2-yloxy)-ethoxy]-1,3-dioxa-6-aza-2-phospha-phenalene), phosphorous acid 2-amino-2-(diethoxy-phosphanyloxy)-ethyl ester diethyl ester, phosphorous acid 2-cyano-2-(diethoxy-phosphanyloxy)-ethyl ester diethyl ester, phosphorous acid 2-(diethoxy-phosphanyloxy)-2-mercapto-ethyl ester diethyl ester, phosphorous acid 2-(diethoxy-phosphanyloxy)-butyl ester diethyl ester, phosphorous acid 2-(diethoxy-phosphanyloxy)-1-methyl-propyl ester diethyl ester, phosphorous acid 2-(diethoxy-phosphanyloxy)-2-ethoxy-ethyl ester diethyl ester, phosphorous acid 2-(tert-butyl-dimethyl-silanyl)-2-(diethoxy-phosphanyloxy)-ethyl ester diethyl ester, phosphorous acid 2-(diethoxy-phosphanyloxy)-2-trimethylsilanyloxy-ethyl ester diethyl ester, phosphorous acid 2-(diethoxy-phosphanyloxy)-2-ethylsulfanyl-ethyl ester diethyl ester, phosphorous acid 2-(diethoxy-phosphanyloxy)-2-(4,4,5,5-tetramethyl-[1,3,2]dioxaborolan-2-yl)-ethyl ester diethyl ester, carbonic acid 1,2-bis-(diethoxy-phosphanyloxy)-ethyl ester methyl ester, acetic acid 1,2-bis-(diethoxy-phosphanyloxy)-ethyl ester, and compounds represented by the following Chemical Formulae, in which iPr denotes isopropyl,

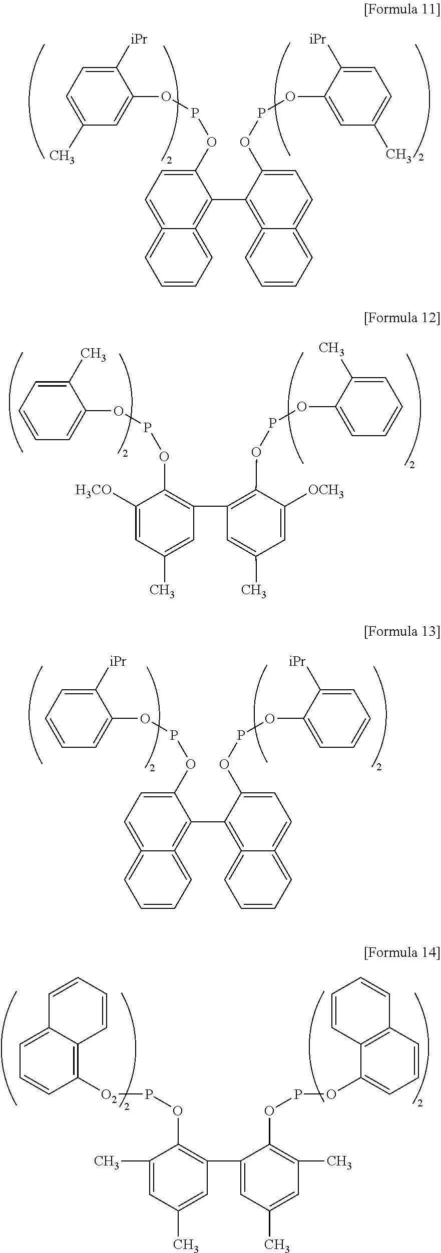

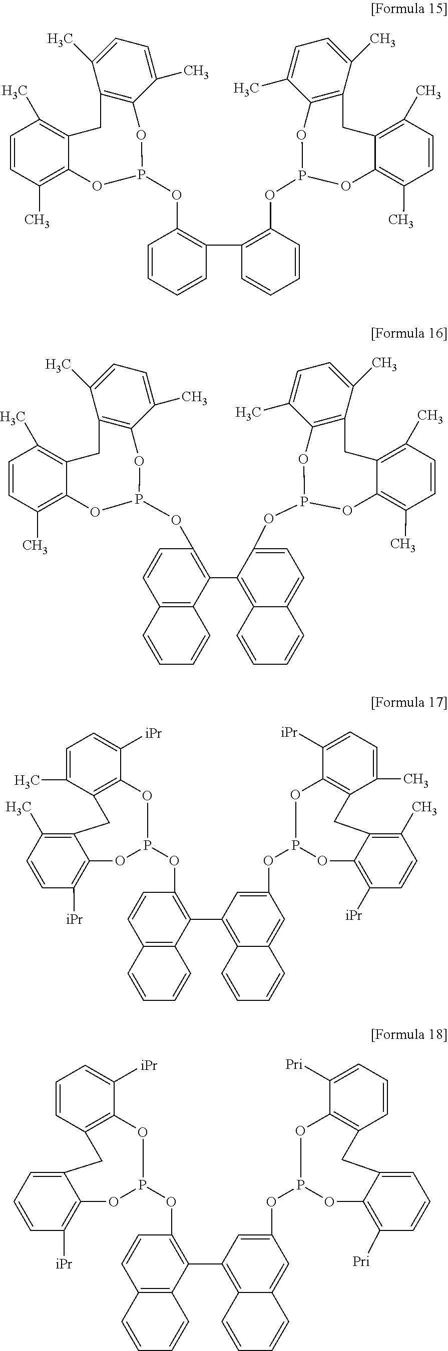

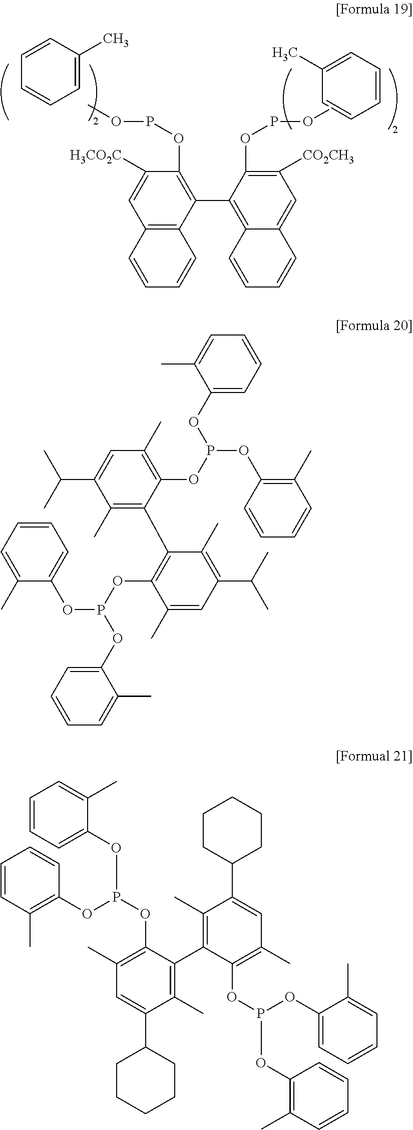

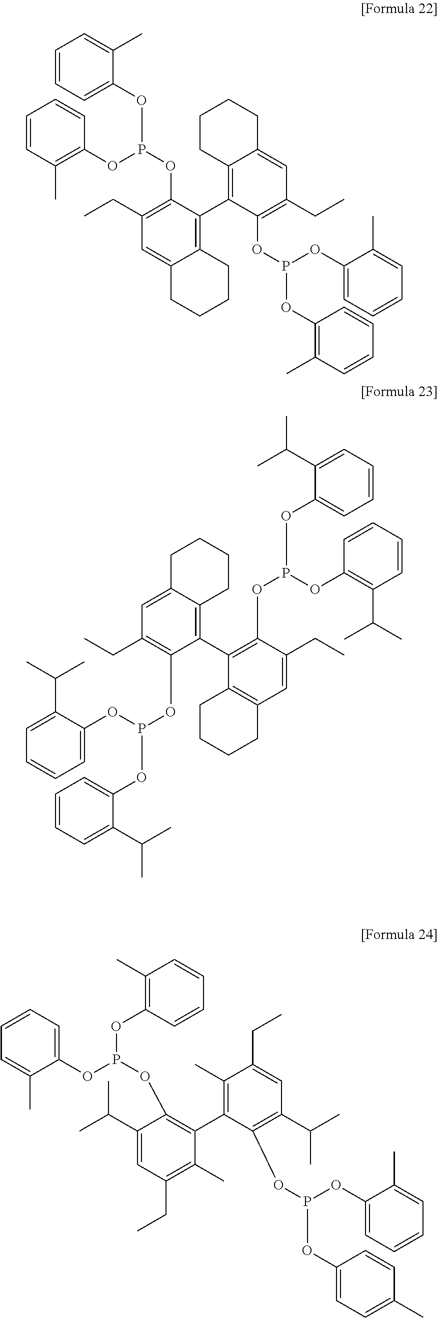

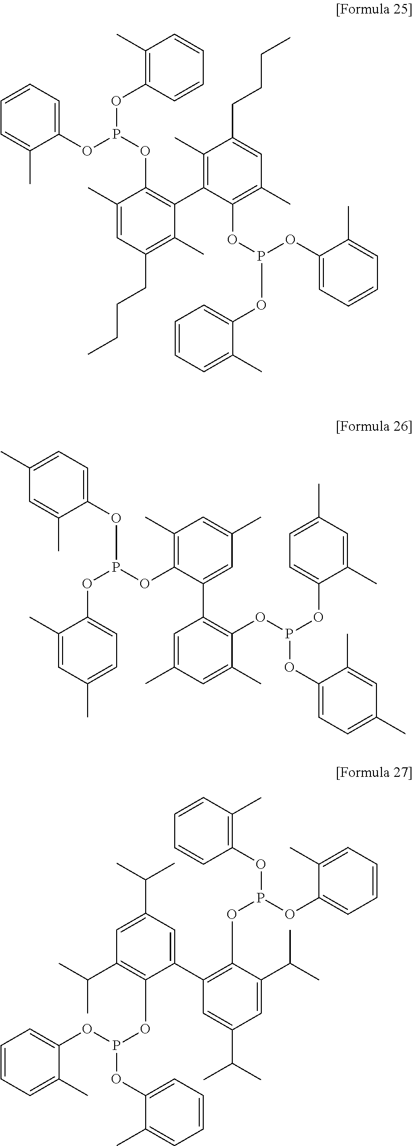

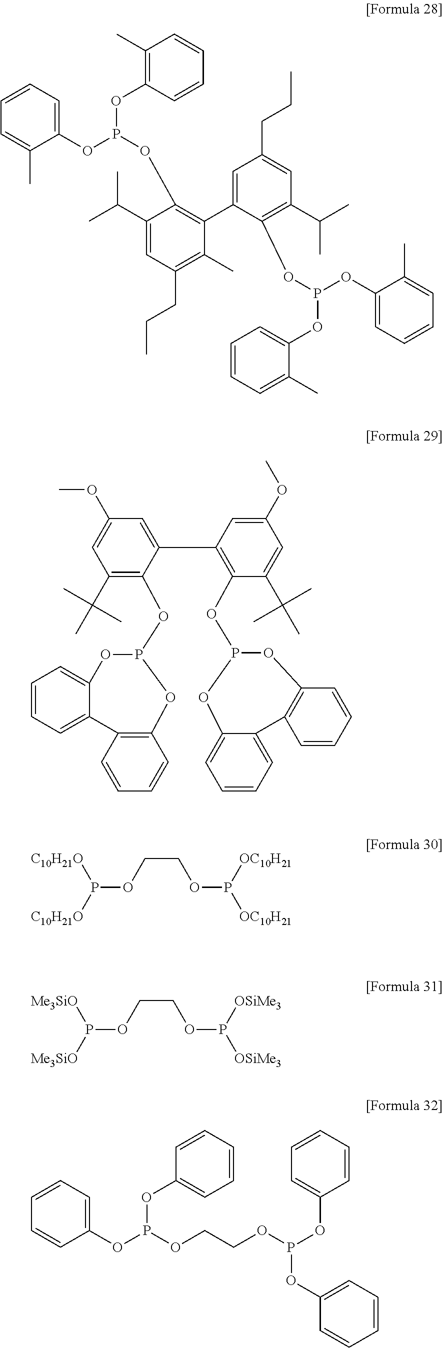

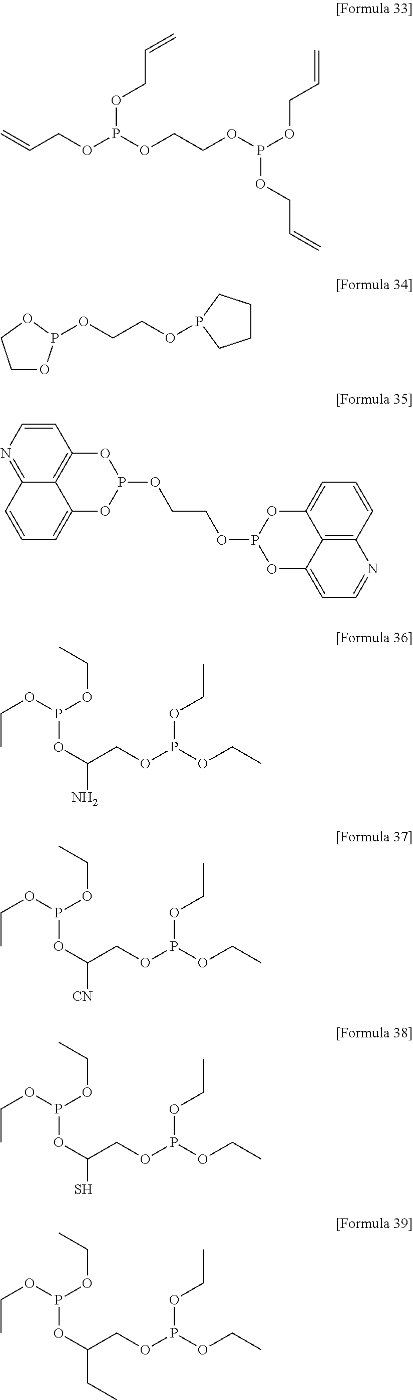

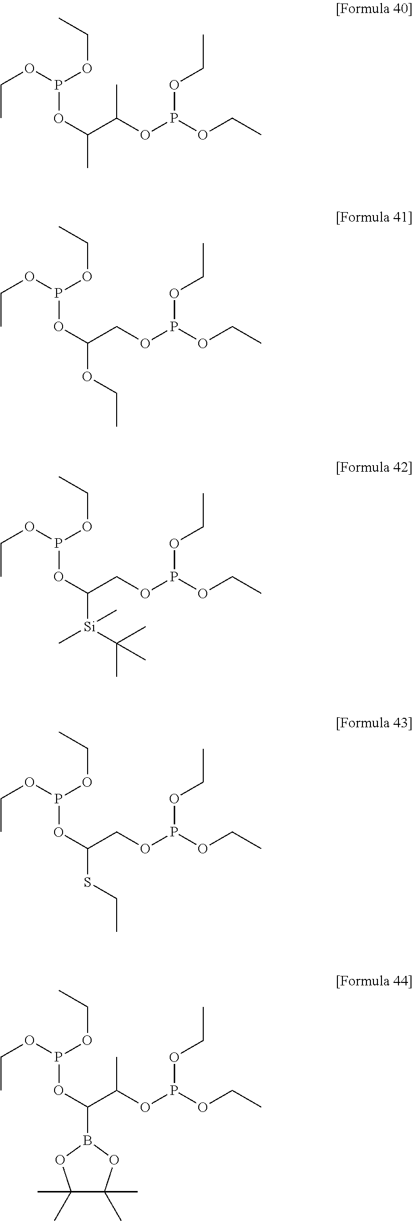 [SMILES: CCOP(OCC)OC(C)C(C)OP(OCC)OCC ; CCOC(COP(OCC)OCC)OP(OCC)OCC ; CCOP(OCC)OCC(OP(OCC)OCC)[Si](C)(C)C(C)(C)C ; CCOP(OCC)OCC(OP(OCC)OCC)SCC ; CCOP(OCC)OC(C)C(OP(OCC)OCC)B1OC(C)(C)C(C)(C)O1]

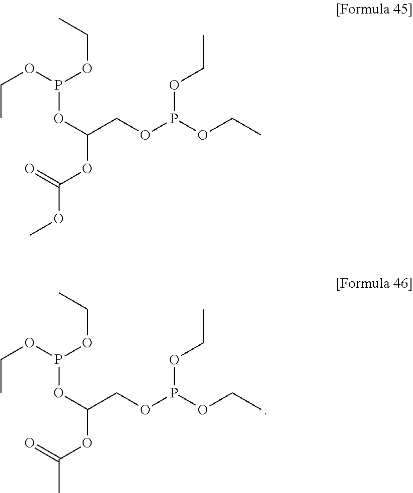 [SMILES: CCOP(OCC)OCC(OC(=O)OC)OP(OCC)OCC ; CCOP(OCC)OCC(OC(C)=O)OP(OCC)OCC]

16. The non-aqueous electrolyte additive as claimed in claim 12, wherein the compound represented by Chemical Formula 1 includes at least one of 6,6′-[(3,3′-di-t-butyl)-5,5′-dimethoxy-1,1′-biphenyl-2,2′-diyl)bis(oxy)]bis(dibenzo[d,f][1,3,2]dioxaphosphepin)hemi ethyl acetate) and 1,4-bis(1,3,2-dioxaphosphacyclopentyl)-1,4-dioxabutane.
